(19) United States
(12) Patent Application Publication
(10) Pub. No.: US 2022/0275461 A1
(43) Pub. Date: Sep. 1, 2022

(54) DIAGNOSTICS AND METHODS FOR PROGNOSING RESPONSE TO IMMUNOTHERAPY BASED ON THE METHYLATION STATUS OF IMMUNE SYNAPSE GENE SIGNATURE

(71) Applicant: H. LEE MOFFITT CANCER CENTER AND RESEARCH INSTITUTE, INC., Tampa, FL (US)

(72) Inventors: Sungjune KIM, Tampa, FL (US); Anders BERGLUND, Tampa, FL (US)

(21) Appl. No.: 17/636,532

(22) PCT Filed: Aug. 21, 2020
(86) PCT No.: PCT/US2020/047475

§ 371 Date: Feb. 18, 2022

Related U.S. Application Data
(60) Provisional application No. 62/889,981, filed on Aug. 21, 2019.

(51) Int. Cl. C12Q 1/6886; A61K 31/706; A61K 31/7068; A61K 39/395; A61P 35/00

(52) U.S. Cl. CPC C12Q 1/6886; A61K 31/706; A61K 31/7068; A61K 39/3955; A61P 35/00; C12Q 2600/106; C12Q 2600/154

(57) ABSTRACT

Disclosed are methods for using the methylation status of a cancerous tissue to assess the susceptibility of a cancer to immunotherapy and determine new treatment regimens. Disclosed are methods related to producing an immunotherapeutic regimen based on the amount of methylation in co-stimulatory genes and/or immune checkpoint genes, as well as, methods of treating an immunogenic cancer based on the same.

### II. PRIORITY CLAIMS

This application claims the benefit of U.S. Provisional Application No. 62/889,981, filed on Aug. 21, 2019, which is incorporated herein by reference in its entirety.

### I. STATEMENT OF GOVERNMENT SUPPORT

This invention was made with government support under Grant No. CA076292 awarded by National Cancer Institute. The government has certain rights in the invention.

### III. BACKGROUND

Cancer Immunotherapy has emerged as newest weapon in the arsenal to combat cancer. Nevertheless, the immunosuppressive effect induced by the tumor microenvironment represents a major obstacle for the success of promising T cell-based immunotherapies, including tumor-expanded T cells, chimeric antigen receptors (CAR)-T cells, and chimeric endocrine receptor (CER)-T cells. One of the obstacles in this field is the inability to predict treatment efficacy and patient response to immunotherapy. Thus, what are needed are new methods to determine the suitability of a subject for an immunotherapy prior to administration of the therapy.

### IV. SUMMARY

Disclosed are methods related to producing an immunotherapeutic regimen based on the amount of methylation in co-stimulatory genes and/or immune checkpoint genes, as well as, methods of treating an immunogenic cancer based on the same.

In one aspect, disclosed herein are methods of treating, inhibiting, reducing, ameliorating, and/or preventing an immunogenic cancer or metastasis (such as, for example, adenocarcinoma, breast cancer, bladder cancer, cervical cancer, colon cancer, lymphoma, esophageal cancer, renal cancer, lung cancer, mesothelioma, head and neck cancer, cholangiocarcinoma, liver cancer, ovarian cancer, pancreatic cancer, prostate cancer, adrenal gland cancer, nerve cell cancer, rectal cancer, melanoma, sarcoma, testicular cancer, thyroid cancer, uterine cancer, or ocular cancer, such cancers including, but not limited to adrenocortical carcinoma, bladder urothelial carcinoma, breast invasive carcinoma, cervical squamous cell carcinoma and endocervical adenocarcinoma, cholangiocarcinoma, colon adenocarcinoma, lymphoid neoplasm diffuse large B-cell lymphoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ sell tumors, thyroid carcinoma, thymoma, uterine corpus endometrial carcinoma, uterine carcinosarcoma, or uveal melanoma) in a subject comprising a) obtaining a tissue sample from the subject; b) assaying the amount of methylation of one or more co-stimulatory genes (such as, for example, cluster of differentiation (CD) 40 (CD40), CD70, homologous to lymphotoxin, exhibits inducible expression and competes with HSV glycoprotein D for binding to herpesvirus entry mediator, a receptor expressed on T lymphocytes (LIGHT), OX40L, CD137L (4-1BBL), glucocorticoid-induced tumour-necrosis-factor-receptor-related protein (GITR) ligand (GITRL), B7 related protein 1 (B7RP1), and/or human leukocyte antigen (HLA)-A (HLA-A)) and/or one or more immune checkpoint genes (such as, for example, carcinoembryonic antigen-related adhesion molecule (CEACAM) 1 (CEACAM1), Galectin 9, programmed death ligand (PDL) 1 (PDL1), PDL2, V-domain Ig suppressor of T cell activation (VISTA), B7-H3, B7-H4, B7-2 (CD86), B7-1 (CD80), HHLA2, CD155, and/or Galectin 3) in the tissue sample; and c) administering to the subject an immunotherapy wherein an increase in the methylation of one or more co-stimulatory genes relative to a normal control tissue is detected and/or a decrease in the methylation of one or more immune checkpoint genes relative to a normal control tissue is detected.

Also disclosed herein are methods treating, inhibiting, reducing, ameliorating, and/or preventing an immunogenic cancer or metastasis of any preceding aspect, wherein the immunotherapy comprises an antibody, cytokine, natural killer (NK) cell, chimeric antigen receptor (CAR) T cell, CAR NK cell, tumor infiltrating lymphocyte (TIL), marrow infiltrating lymphocyte (MIL), and/or tumor infiltrating NK cell (TINK), for example, an immune checkpoint inhibitor blockade.

In one aspect, disclosed herein are method treating, inhibiting, reducing, ameliorating, and/or preventing an immunogenic cancer or metastasis of any preceding aspect, further comprising administering to the subject an inhibitor of methylation (such as, for example, azacytidine, decitabine, and/or zebularine) when the amount of methylation of the one or more co-stimulatory genes is increased relative to a normal tissue control or not administering to the subject an inhibitor of methylation (such as, for example, azacytidine, decitabine, and/or zebularine) when the amount of methylation of the one or more co-stimulatory genes is decreased relative to a normal tissue control or the amount of methylation of the one or more immune checkpoint genes is increased relative to a normal tissue control.

Also disclosed herein are methods of treating, inhibiting, reducing, ameliorating, and/or preventing an immunogenic cancer or metastasis of any preceding aspect, wherein methylation is measured by performing principal component (PC) analysis (PCA) of the one or more co-stimulatory genes and/or one or more immune checkpoint genes; wherein PC^{high} indicates an increase in methylation and PC^{low} indicates a decrease in methylation.

In one aspect, disclosed herein are methods of assessing the suitability of an immunotherapy treatment regimen for the treatment an immunogenic cancer or metastasis (such as, for example, adenocarcinoma, breast cancer, bladder cancer, cervical cancer, colon cancer, lymphoma, esophageal cancer, renal cancer, lung cancer, mesothelioma, head and neck cancer, cholangiocarcinoma, liver cancer, ovarian cancer, pancreatic cancer, prostate cancer, adrenal gland cancer, nerve cell cancer, rectal cancer, melanoma, sarcoma, testicular cancer, thyroid cancer, uterine cancer, or ocular cancer, such cancers including, but not limited to adrenocortical carcinoma, bladder urothelial carcinoma, breast invasive carcinoma, cervical squamous cell carcinoma and endocervical adenocarcinoma, cholangiocarcinoma, colon adenocarcinoma, lymphoid neoplasm diffuse large B-cell lymphoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ sell tumors, thyroid carcinoma, thymoma, uterine corpus endometrial carcinoma, uterine carcinosarcoma, or uveal melanoma) in a subject comprising a) obtaining a tissue sample from the subject; and b) assaying the amount of methylation of one or more co-stimulatory genes (such as, for example, cluster of differentiation (CD) 40 (CD40), CD70, homologous to lymphotoxin, exhibits inducible expression and competes with HSV glycoprotein D for binding to herpesvirus entry mediator, a receptor expressed on T lymphocytes (LIGHT), OX40L, CD137L (4-1BBL), glucocorticoid-induced tumour-necrosis-factor-receptor-related protein (GITR) ligand (GITRL), B7 related protein 1(B7RP1), and/or human leukocyte antigen (HLA)-A (HLA-A)) and/or one or more immune checkpoint genes (such as, for example, carcinoembryonic antigen-related adhesion molecule (CEACAM) 1 (CEACAM1), Galectin 9, programmed death ligand (PDL) 1 (PDL1), PDL2, V-domain Ig suppressor of T cell activation (VISTA), B7-H3, B7-H4, B7-2 (CD86), B7-1 (CD80), HHLA2, CD155, and/or Galectin 3) in the tissue sample; wherein an increase in the methylation of one or more co-stimulatory genes relative to a normal control tissue and/or a decrease in the methylation of one or more immune checkpoint genes relative to a normal control tissue indicates that immunotherapy is suitable for treatment of the cancer in the subject.

Also disclosed herein are methods of assessing the suitability of an immunotherapy treatment regimen for the treatment of an immunogenic cancer or metastasis in a subject of any preceding aspect, wherein the immunotherapy comprises an antibody, cytokine, natural killer (NK) cell, chimeric antigen receptor (CAR) T cell, CAR NK cell, tumor infiltrating lymphocyte (TIL), marrow infiltrating lymphocyte (MIL), and/or tumor infiltrating NK cell (TINK)), for example, an immune checkpoint inhibitor blockade.

In one aspect, disclosed herein are methods of assessing the suitability of an immunotherapy treatment regimen for the treatment of an immunogenic cancer or metastasis in a subject of any preceding aspect, wherein a decrease in the methylation of one or more co-stimulatory genes relative to a normal control tissue or an increase in the methylation of one or more immune checkpoint genes relative to a normal control indicates that an inhibitor of methylation should not be administered to the subject.

Also disclosed herein are methods of assessing the suitability of an immunotherapy treatment regimen for the treatment of an immunogenic cancer or metastasis in a subject of any preceding aspect, wherein an increase in the methylation of one or more co-stimulatory genes relative to a normal control tissue indicates that an inhibitor of methylation can be administered to the subject.

In one aspect, disclosed herein are methods of assessing the suitability of an immunotherapy treatment regimen for the treatment of an immunogenic cancer or metastasis in a subject of any preceding aspect, wherein the assessment is conducted prior to the commencement of any immunotherapy regimen; wherein an increase in the methylation of one or more co-stimulatory genes relative to a normal control tissue and/or a decrease in the methylation of one or more immune checkpoint genes relative to a normal control tissue indicates that the subject can start an immunotherapy regimen; and wherein a decrease in the methylation or same amount of methylation of one or more co-stimulatory genes relative to a normal control tissue and/or an increase in the methylation of one or more immune checkpoint genes relative to a normal control tissue indicates that the subject should start an anti-cancer regimen that is not an immunotherapy.

Also disclosed herein are methods of assessing the suitability of an immunotherapy treatment regimen for the treatment of an immunogenic cancer or metastasis in a subject of any preceding aspect, wherein the assessment is conducted after to the commencement of an immunotherapy regimen; wherein an increase in the methylation of one or more co-stimulatory genes relative to a normal control tissue and/or a decrease in the methylation of one or more immune checkpoint genes relative to a normal control tissue indicates that the subject can continue an immunotherapy regimen; and wherein a decrease or same amount of methylation of one or more co-stimulatory genes relative to a normal control tissue and/or an increase or same amount of methylation of one or more immune checkpoint genes relative to a normal control tissue indicates that the subject should discontinue an anti-cancer regimen that is not an immunotherapy.

In one aspect, disclosed herein are methods of assessing the suitability of an immunotherapy treatment regimen for the treatment of an immunogenic cancer or metastasis in a subject of any preceding aspect, wherein methylation is measured by performing principal component analysis of the one or more co-stimulatory genes and/or one or more immune checkpoint genes; wherein PC^{high} indicates an increase in methylation and PC^{low} indicates a decrease in methylation.

### V. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, and 1D show the distinct pattern of immune synapse gene methylation depends on tumor histology. FIG. 1A shows the schematic of immune synapse between the antigen presenting cells/tumor and T-cells is demonstrated. FIG. 1B shows T-SNE analysis was performed on 8,186 solid tumors and 745 normal adjacent tissues based on the β-values for methylation levels on all probes for CSGs and ICGs from (1A) contrasting tumor (blue) vs. normal adjacent tissue (red). FIG. 1C shows the spatial relationship between distinct tumor types is depicted with breast tumors in blue- and normal adjacent tissue samples black-dotted boxes. FIG. 1D shows unbiased hierarchical clustering analysis is shown.

FIGS. 2A, 2B, 2C, 2D, 2F, 2G, and 2H show the polarity of methylation patterns for co-stimulatory and immune checkpoint ligands. FIGS. 2A and 2B show β-values of methylation probes for TSS1500, TSS200, 5′UTR, body, and 3′UTR of HHLA2 gene (2A), an example of ICG, or CD40 (2B), an example of CSG, derived from all tumor samples (blue) and normal adjacent tissues (red) are depicted. The methylation level for each probe is represented by a box-plot. The left most column indicates the presence of CpG-island, while the second column colors indicate where on the gene the probe is located. The genomic location is listed on the left y-axis, probe id on the right y-axis and probes selected for further analysis is marked with blue color. FIGS. 2C and 2D show a heatmap of the correlation coefficient between all the probes within a gene, HHLA2 (2C) or CD40 (2D) across all tumor types. FIGS. 2E and 2F show a box plot of average β-values for selected probes HHLA2 (2E) or CD40 (2F) from tumor (blue) and normal adjacent tissue (red) are shown. FIGS. 2G and 2H show the average β-values for selected probes within TSS1500, TSS200, and 5′UTR are plotted against gene expression for HHLA2 (2C) or CD40 (2F). Each circle represents an individual tissue sample.

FIGS. 3A, 3B, 3C, and 3D show the methylation pattern for CEACAM1. FIG. 3A shows β-values of methylation probes for TSS1500, TSS200, 5′UTR, body, and 3′UTR of CEACAM1 gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 3B shows a heatmap of the correlation coefficient between all the probes within a gene across all tumor types. FIG. 3C show the average β-values for selected probes within TSS1500, TSS200, and 5′UTR are plotted against gene expression. Each circle represents an individual tissue sample. FIG. 3D shows a box plot of average β-values for selected probes from tumor (blue) and normal adjacent tissue (red) are shown.

FIGS. 4A, 4B, 4C, and 4D show the methylation pattern for LGALS9 (Galectin9). FIG. 4A shows β-values of methylation probes for TSS1500, TSS200, 5′UTR, body, and 3′UTR of LGALS9 (Galectin9) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 4B shows a heatmap of the correlation coefficient between all the probes within a gene across all tumor types. FIG. 4C shows the average β-values for selected probes within TSS1500, TSS200, and 5′UTR are plotted against gene expression. Each circle represents an individual tissue sample. FIG. 4D shows a box plot of average β-values for selected probes from tumor (blue) and normal adjacent tissue (red) are shown.

FIGS. 5A, 5B, 5C, and 5D show the methylation pattern for CD274 (PDL1). FIG. 5A shows β-values of methylation probes for TSS1500, TSS200, 5′UTR, body, and 3′UTR of CD274 (PDL1) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 5B shows a heatmap of the correlation coefficient between all the probes within a gene across all tumor types. FIG. 5C shows the average β-values for selected probes within TSS1500, TSS200, and 5′UTR are plotted against gene expression. Each circle represents an individual tissue sample. FIG. 5D shows a box plot of average β-values for selected probes from tumor (blue) and normal adjacent tissue (red) are shown.

FIGS. 6A, 6B, 6C, and 6D show the methylation pattern for PDCD1LG2 (PDL2). FIG. 6A shows β-values of methylation probes for TSS1500, TSS200, 5′UTR, body, and 3′UTR of PDCD1LG2 (PDL2) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 6B shows a heatmap of the correlation coefficient between all the probes within a gene across all tumor types. FIG. 6C shows the average β-values for selected probes within TSS1500, TSS200, and 5′UTR are plotted against gene expression. Each circle represents an individual tissue sample. FIG. 6D shows a box plot of average β-values for selected probes from tumor (blue) and normal adjacent tissue (red) are shown.

FIGS. 7A, 7B, 7C, and 7D show the methylation pattern for C10orf54 (VISTA). FIG. 7A shows β-values of methylation probes for TSS1500, TSS200, 5′UTR, body, and 3′UTR of C10orf54 (VISTA) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 7B shows a heatmap of the correlation coefficient between all the probes within a gene across all tumor types. FIG. 7C shows the average β-values for selected probes within TSS1500, TSS200, and 5′UTR are plotted against gene expression. Each circle represents an individual tissue sample. FIG. 7D shows a box plot of average β-values for selected probes from tumor (blue) and normal adjacent tissue (red) are shown.

FIGS. 8A, 8B, 8C, and 8D show the methylation pattern for CD276 (B7-H3). FIG. 8A shows β-values of methylation probes for TSS1500, TSS200, 5′UTR, body, and 3′UTR of CD276 (B7-H3) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 8B shows a heatmap of the correlation coefficient between all the probes within a gene across all tumor types. FIG. 8C shows the average β-values for selected probes within TSS1500, TSS200, and 5′UTR are plotted against gene expression. Each circle represents an individual tissue sample. FIG. 8D shows a box plot of average β-values for selected probes from tumor (blue) and normal adjacent tissue (red) are shown.

FIGS. 9A, 9B, 9C, and 9D show the methylation pattern for VTCN1 (B7-H4). FIG. 9A shows β-values of methylation probes for TSS1500, TSS200, 5′UTR, body, and 3′UTR of VTCN1 (B7-H4) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 9B shows a heatmap of the correlation coefficient between all the probes within a gene across all tumor types. FIG. 9C shows the average β-values for selected probes within TSS1500, TSS200, and 5′UTR are plotted against gene expression. Each circle represents an individual tissue sample. FIG. 9D shows a box plot of average β-values for selected probes from tumor (blue) and normal adjacent tissue (red) are shown.

FIGS. 10A, 10B, 10C, and 10D show the methylation pattern for CD86. FIG. 10A shows β-values of methylation probes for TSS1500, TSS200, 5′UTR, body, and 3′UTR of CD86 gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 10B shows a heatmap of the correlation coefficient between all the probes within a gene across all tumor types. FIG. 10C shows the average β-values for selected probes within TSS1500, TSS200, and 5′UTR are plotted against gene expression. Each circle represents an individual tissue sample. FIG. 10D shows a box plot of average β-values for selected probes from tumor (blue) and normal adjacent tissue (red) are shown.

FIGS. 11A, 11B, 11C, and 11D show the methylation pattern for CD80. FIG. 11A shows β-values of methylation probes for TSS1500, TSS200, 5′UTR, body, and 3′UTR of CD80 gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 11B shows a heatmap of the correlation coefficient between all the probes within a gene across all tumor types. FIG. 11C shows the average β-values for selected probes within TSS1500, TSS200, and 5′UTR are plotted against gene expression. Each circle represents an individual tissue sample. FIG. 11D shows a box plot of average β-values for selected probes from tumor (blue) and normal adjacent tissue (red) are shown.

FIGS. 12A, 12B, 12C, and 12D show the methylation pattern for PVR. FIG. 12A shows β-values of methylation probes for TSS1500, TSS200, 5′UTR, body, and 3′UTR of PVR gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 12B shows a heatmap of the correlation coefficient between all the probes within a gene across all tumor types. FIG. 12C shows the average β-values for selected probes within TSS1500, TSS200, and 5′UTR are plotted against gene expression. Each circle represents an individual tissue sample. FIG. 12D shows a box plot of average β-values for selected probes from tumor (blue) and normal adjacent tissue (red) are shown.

FIGS. 13A, 13B, 13C, and 13D show the methylation pattern for LGALS3 (Galectin3). FIG. 13A shows β-values of methylation probes for TSS1500, TSS200, 5′UTR, body, and 3′UTR of LGALS3 (Galectin3) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 13B shows a heatmap of the correlation coefficient between all the probes within a gene across all tumor types. FIG. 13C shows the average β-values for selected probes within TSS1500, TSS200, and 5′UTR are plotted against gene expression. Each circle represents an individual tissue sample. FIG. 13D shows a box plot of average β-values for selected probes from tumor (blue) and normal adjacent tissue (red) are shown.

FIGS. 14A, 14B, 14C, and 14D show the methylation pattern for TNFSF14 (LIGHT). FIG. 14A shows β-values of methylation probes for TSS1500, TSS200, 5′UTR, body, and 3′UTR of TNFSF14 (LIGHT) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 14B shows a heatmap of the correlation coefficient between all the probes within a gene across all tumor types. FIG. 14C shows the average β-values for selected probes within TSS1500, TSS200, and 5′UTR are plotted against gene expression. Each circle represents an individual tissue sample. FIG. 14D shows a box plot of average β-values for selected probes from tumor (blue) and normal adjacent tissue (red) are shown.

FIGS. 15A, 15B, 15C, and 15D show the methylation pattern for TNFSF4 (OX40L). FIG. 15A shows β-values of methylation probes for TSS1500, TSS200, 5′UTR, body, and 3′UTR of TNFSF4 (OX40L) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 15B shows a heatmap of the correlation coefficient between all the probes within a gene across all tumor types. FIG. 15C shows the average β-values for selected probes within TSS1500, TSS200, and 5′UTR are plotted against gene expression. Each circle represents an individual tissue sample. FIG. 15D shows a box plot of average β-values for selected probes from tumor (blue) and normal adjacent tissue (red) are shown.

FIGS. 16A, 16B, 16C, and 16D show the methylation pattern for TNFSF9 (CD173L). FIG. 16A shows β-values of methylation probes for TSS1500, TSS200, 5′UTR, body, and 3′UTR of TNFSF9 (CD173L) gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 16B shows a heatmap of the correlation coefficient between all the probes within a gene across all tumor types. FIG. 16C shows the average β-values for selected probes within TSS1500, TSS200, and 5′UTR are plotted against gene expression. Each circle represents an individual tissue sample. FIG. 16D shows a box plot of average β-values for selected probes from tumor (blue) and normal adjacent tissue (red) are shown.

FIGS. 17A, 17B, 17C, and 17D show the methylation pattern for HLA-A. FIG. 17A shows β-values of methylation probes for TSS1500, TSS200, 5′UTR, body, and 3′UTR of HLA-A gene derived from all tumor samples (blue) and normal adjacent tissues (red) are depicted. The methylation level for each probe is represented by a box-plots. The left most column indicates the presence of CpG-island, while the second column colors indicate where on the gene the probe is located. The genomic location is listed on the left y-axis and the probe id is shown on the right y-axis the probes selected for further analysis is marked with blue color. FIG. 17B shows a heatmap of the correlation coefficient between all the probes within a gene across all tumor types. FIG. 17C shows the average β-values for selected probes within TSS1500, TSS200, and 5′UTR are plotted against gene expression. Each circle represents an individual tissue sample. FIG. 17D shows a box plot of average β-values for selected probes from tumor (blue) and normal adjacent tissue (red) are shown.

FIGS. 18A, 18B, 18C, 18D, 18E, and 18F show the principal component analysis (PCA) segregates co-stimulatory and immune checkpoint ligands. FIG. 18A shows two-dimensional plot of PC1 and PC2 scores for all tumor types (blue) and normal adjacent tissues (red) is shown. FIG. 18B shows the importance of each variable, CpG-probes, for PC1 and PC2 are depicted. A box plot of PC1 (18C) and PC2 (18D) scores for tumor (blue) and normal adjacent tissue (red) compared across histologic types. FIG. 18E shows PC1 scores of mock- or 5-azacitidine-treated epithelial cancer cell lines. FIGS. 18F shows the methylation status of CD40 gene in mock- or azacytidine-treated CAMA1 cell line. * p<0.05, ** p<0.01, *** p<0.001, **** p<0.0001 by t-test.

FIGS. 19A, 19B, 19C, 19D, and 19E show the correlation of PC1 and PC2 with CSG and ICG. FIG. 19A shows a scatter plot of PC1 score vs. average β-values of CSG probes is shown. FIG. 19B shows a scatter plot of PC2 score vs. average β-values of ICG probes is shown. FIGS. 19C shows the PCA loadings of each variable, CpG-probes, for CSG probes (Blue circles) and ICG probes (Red squares) are depicted. A box plot of average β-values of CSG probes (19D) and average β-values of ICG probes (19E) for tumor (blue) and normal adjacent tissue (red) are compared across histologic types.

FIGS. 20A, 20B, 20C, 20D, 20E, 20F, 20G, and 20H show the methylation status of co-stimulatory ligands is prognostic in melanoma. FIG. 20A shows Kaplan-Meier curves for DSS of melanoma patients with high, intermediate, and low tertials of PC1 score are shown. Higher PC1 score represents hypermethylation of CSGs. FIG. 20B shows Box-plot of PC1 score distribution based on melanoma patient staging. FIGS. 20C and 20D show Kaplan-Meier curves for DSS of UCEC patients with MSI (20C) or without MSI (20D) with high, intermediate, and low tertiles of PC1 score are shown. FIG. 20E shows T-cell recruitment in PC1high and PC1low melanoma patients is approximated by gene expression of CD3E, CD4 and CD8B. FIG. 20H shows T effector functions in PC1high and PC1low melanoma patients is approximated by gene expression of CD3ζ(CD247), Granzyme B (GZMB), Perforin (PRF1), and IFNγ. FIG. 20G shows chemokines for immune cell trafficking in PC1high and PC1low melanoma patients is approximated by gene expression of CCL2, CCL3, CCL4, CCLS, CCL9 and CCL10. FIG. 20H shows immunogenicity of PC1high and PC1low melanoma patients is approximated by gene expression of cGAS. p-value in panel 20A, 20C and 20D is a log rank test between High and Low group. ****, p<0.0001 by t-test.

FIG. 21 shows a PLS regression model in melanoma. The PLS model was developed by analysis of patients with longer DSS vs. shorter DSS in the training set in melanoma. Kaplan-Meier curves for DSS of the validation melanoma cohort is depicted based on the high vs. low predicted response by median.

FIGS. 22A, 22B, 22C, 22D, 22E, 22F, and 22G show that PC1 predicts OS and DSS in immunogenic cancers. FIG. 22A shows Kaplan-Meier curves for OS of melanoma patients with high, intermediate, and low tertile of PC1 score are shown. FIGS. 22B and 22C show Kaplan-Meier curves for OS (22B) and DSS (22C) of lung squamous cell carcinoma with high, intermediate and low tertile of PC1 score are shown. FIGS. 22D and 22E show Kaplan-Meier curves for OS (22D) and DSS (22E) of lung adenocarcinoma patients with high, intermediate, and low tertile of PC1 score are shown. FIG. 22F and 22G show Kaplan-Meier curves for OS of uterine cancer patients with MSI (22F) or without MSI (22G) with high, intermediate, and low tertials of PC1 score are shown.

FIGS. 23A, 23B, 23C, 23D, 23E, and 23F show that PC2 predicts OS and DSS in immunogenic cancers. FIGS. 23A and 23B show Kaplan-Meier curves for OS (23A) and DSS (23B) of head and neck squamous cell carcinoma with high, intermediate and low tertile of PC2 score are shown. FIGS. 23C and 23D show Kaplan-Meier curves for OS (23C) and DSS (23D) of renal clear cell carcinoma patients with high, intermediate, and low tertile of PC1 score are shown. FIGS. 23E and 23F show Kaplan-Meier curves for OS (23E) and DSS (23F) of renal papillary carcinoma patients with high, intermediate, and low tertile of PC1 score are shown.

FIGS. 24A and 24B show that PC1 can predict response to immunotherapy in melanoma.

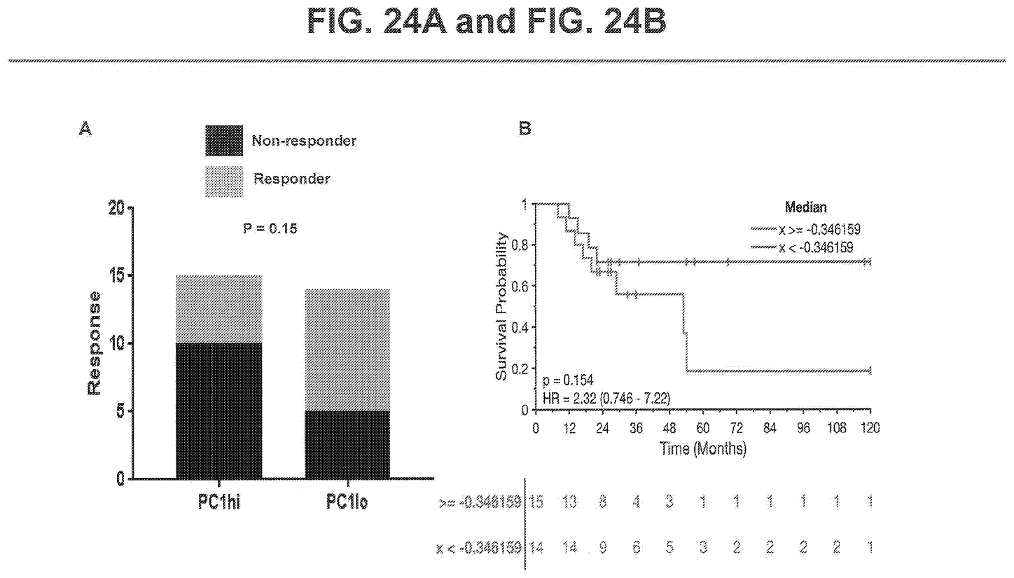

FIG. 25 shows a cartoon showing the co-stimulatory and checkpoint interactions of a T cell and a tumor cell.

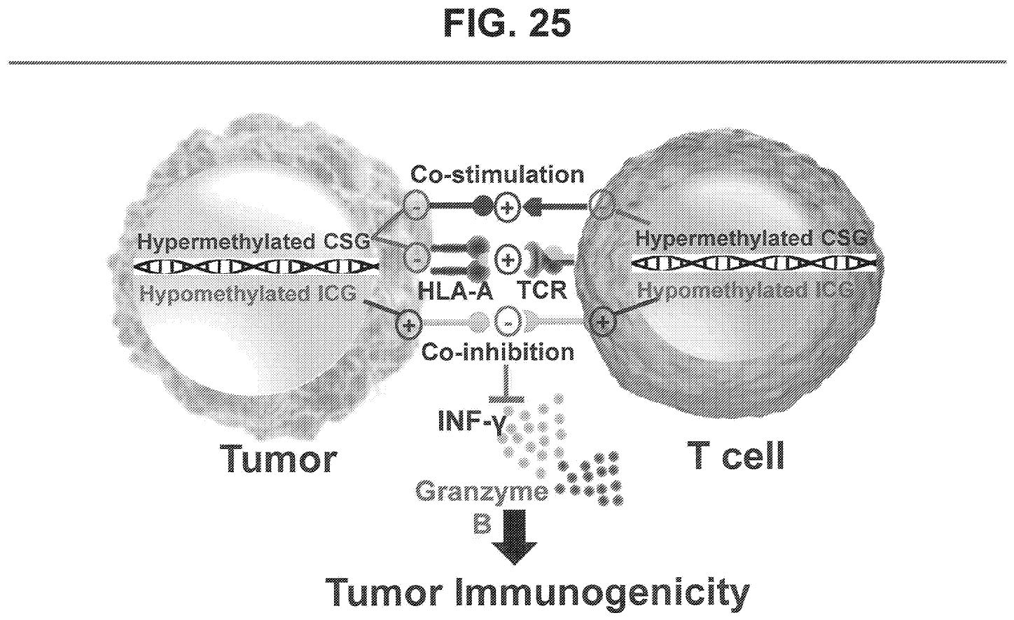

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.” A normal control can refer to a tissue sample that is disease and/or cancer free either obtained from a subject with a cancer (such as a neighboring disease free tissue) or a subject without a cancer.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Treating a Cancer and Assessing a Cancer Treatment Regimen

Cancer immune evasion is achieved through multiple layers of immune tolerance mechanisms including immune editing, recruitment of tolerogenic immune cells, and secretion of immune suppressive cytokines. Recent success with immune checkpoint inhibitors in cancer immunotherapy indicates a dysfunctional immune synapse as a pivotal tolerogenic mechanism. Tumor cells express immune synapse proteins to suppress the immune system, which is often modulated by epigenetic mechanisms. When the methylation status of key immune synapse genes was interrogated, a disproportionately hyper-methylated co-stimulatory genes and hypo-methylation of immune checkpoint genes was observed, which were negatively associated with functional T-cell recruitment to the tumor microenvironment. Therefore, the methylation status of immune synapse genes reflects tumor immunogenicity and correlates with survival.

In one aspect, disclosed herein are methods of treating, inhibiting, reducing, ameliorating, and/or preventing an immunogenic cancer or metastasis (such as, for example, adenocarcinoma, breast cancer, bladder cancer, cervical cancer, colon cancer, lymphoma, esophageal cancer, renal cancer, lung cancer, mesothelioma, head and neck cancer, cholangiocarcinoma, liver cancer, ovarian cancer, pancreatic cancer, prostate cancer, adrenal gland cancer, nerve cell cancer, rectal cancer, melanoma, sarcoma, testicular cancer, thyroid cancer, uterine cancer, or ocular cancer, such cancers including, but not limited to adrenocortical carcinoma, bladder urothelial carcinoma, breast invasive carcinoma, cervical squamous cell carcinoma and endocervical adenocarcinoma, cholangiocarcinoma, colon adenocarcinoma, lymphoid neoplasm diffuse large B-cell lymphoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ sell tumors, thyroid carcinoma, thymoma, uterine corpus endometrial carcinoma, uterine carcinosarcoma, or uveal melanoma) in a subject comprising a) obtaining a tissue sample from the subject; b) assaying the amount of methylation of one or more co-stimulatory genes (such as, for example, cluster of differentiation (CD) 40 (CD40), CD70, homologous to lymphotoxin, exhibits inducible expression and competes with HSV glycoprotein D for binding to herpesvirus entry mediator, a receptor expressed on T lymphocytes (LIGHT), OX40L, CD137L (4-1BBL), glucocorticoid-induced tumour-necrosis-factor-receptor-related protein (GITR) ligand (GITRL), B7 related protein 1 (B7RP1), and/or human leukocyte antigen (HLA)-A (HLA-A)) and/or one or more immune checkpoint genes (such as, for example, carcinoembryonic antigen-related adhesion molecule (CEACAM) 1 (CEACAM1), Galectin 9, programmed death ligand (PDL) 1 (PDL1), PDL2, V-domain Ig suppressor of T cell activation (VISTA), B7-H3, B7-H4, B7-2 (CD86), B7-1 (CD80), HHLA2, CD155, and/or Galectin 3) in the tissue sample; and c) administering to the subject an immunotherapy wherein an increase in the methylation of one or more co-stimulatory genes relative to a normal control tissue is detected and/or a decrease in the methylation of one or more immune checkpoint genes relative to a normal control tissue is detected.

As disclosed herein methylation, and in particular, hypermethylation (i.e., an increase in methylation relative to a normal tissue control) can lead to a decrease in gene expression of co-stimulatory genes which reduces an immune response to a cancer. Thus, decreasing methylation in an hypermethylated of co-stimulatory genes through, for example, the administration of any inhibitor of methylation (such as, for example, azacytidine, decitabine, and/or zebularine) can alone or in combination with an immunotherapy (including, any of the immune checkpoint inhibitor blockades disclosed herein) decrease, inhibit, ameliorate, reduce, treat, and/or prevent a cancer or metastasis. However, administration of an inhibitor of methylation when co-stimulatory genes are hypomethylated and/or immune checkpoint genes are hypermethylated can have a detrimental effect. Thus, in one aspect, disclosed herein are method treating, inhibiting, reducing, ameliorating, and/or preventing an immunogenic cancer or metastasis, further comprising administering to the subject an inhibitor of methylation (such as, for example, azacytidine, decitabine, and/or zebularine) when the amount of methylation of the one or more co-stimulatory genes is increased relative to a normal tissue control or not administering to the subject an inhibitor of methylation (such as, for example, azacytidine, decitabine, and/or zebularine) when the amount of methylation of the one or more co-stimulatory genes is decreased relative to a normal tissue control or the amount of methylation of the one or more immune checkpoint genes is increased relative to a normal tissue control.

As disclosed herein methylation of co-stimulatory genes and/or immune checkpoint genes can have a significant effect on the efficacy of immunotherapy. Applying immunotherapy to a cancer in a subject that has the wrong methylation profile will not only not be effective, but ultimately decreases the likelihood of successful treatment as the cancer will have an opportunity to grow and spread while the ineffective immunotherapy is being applied. Knowing that a cancer is not susceptible to immunotherapy before administration or being able to assess a cancer and stope an immunotherapy treatment in a subject has profound benefit to the patient as more successful treatment options could be used instead. Alternatively, knowing that a cancer is susceptible to immunotherapy can guide the caregiver to administer an immunotherapy early or stay the course if already implemented. Thus, in one aspect, disclosed herein are methods of assessing the suitability of an immunotherapy treatment regimen for the treatment an immunogenic cancer or metastasis (such as, for example, adenocarcinoma, breast cancer, bladder cancer, cervical cancer, colon cancer, lymphoma, esophageal cancer, renal cancer, lung cancer, mesothelioma, head and neck cancer, cholangiocarcinoma, liver cancer, ovarian cancer, pancreatic cancer, prostate cancer, adrenal gland cancer, nerve cell cancer, rectal cancer, melanoma, sarcoma, testicular cancer, thyroid cancer, uterine cancer, or ocular cancer, such cancers including, but not limited to adrenocortical carcinoma, bladder urothelial carcinoma, breast invasive carcinoma, cervical squamous cell carcinoma and endocervical adenocarcinoma, cholangiocarcinoma, colon adenocarcinoma, lymphoid neoplasm diffuse large B-cell lymphoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ sell tumors, thyroid carcinoma, thymoma, uterine corpus endometrial carcinoma, uterine carcinosarcoma, or uveal melanoma) in a subject comprising a) obtaining a tissue sample from the subject; and b) assaying the amount of methylation of one or more co-stimulatory genes (such as, for example, cluster of differentiation (CD) 40 (CD40), CD70, homologous to lymphotoxin, exhibits inducible expression and competes with HSV glycoprotein D for binding to herpesvirus entry mediator, a receptor expressed on T lymphocytes (LIGHT), OX40L, CD137L (4-1BBL), glucocorticoid-induced tumour-necrosis-factor-receptor-related protein (GITR) ligand (GITRL), B7 related protein 1 (B7RP1), and/or human leukocyte antigen (HLA)-A (HLA-A)) and/or one or more immune checkpoint genes (such as, for example, carcinoembryonic antigen-related adhesion molecule (CEACAM) 1 (CEACAM1), Galectin 9, programmed death ligand (PDL) 1 (PDL1), PDL2, V-domain Ig suppressor of T cell activation (VISTA), B7-H3, B7-H4, B7-2 (CD86), B7-1 (CD80), HHLA2, CD155, and/or Galectin 3) in the tissue sample; wherein an increase in the methylation of one or more co-stimulatory genes relative to a normal control tissue and/or a decrease in the methylation of one or more immune checkpoint genes relative to a normal control tissue indicates that immunotherapy is suitable for treatment of the cancer in the subject.

It is understood and herein contemplated that the disclosed immunotherapy treatment assessment methods are useful both prior to any administration of an immunotherapy to tell of the immunotherapy should or should not be administered and also after commencement of an immunotherapy to determine if said immunotherapy should be continued. Knowing the methylation status allows the treating physician to avoid wasting valuable treatment time or unnecessarily subjecting the patient to an ineffective therapy by discontinuing an immunotherapy or never starting immunotherapy if the cancer does not have the correct methylation profile for the immunotherapy to be efficacious. Alternatively, where the methylation profile is appropriate, immunotherapy can be initiated or continued. Thus, in one aspect, disclosed herein are methods of assessing the suitability of an immunotherapy treatment regimen for the treatment of an immunogenic cancer or metastasis in a subject, wherein the assessment is conducted prior to the commencement of any immunotherapy regimen; wherein an increase in the methylation of one or more co-stimulatory genes relative to a normal control tissue and/or a decrease in the methylation of one or more immune checkpoint genes relative to a normal control tissue indicates that the subject can start an immunotherapy regimen; and wherein a decrease in the methylation or same amount of methylation of one or more co-stimulatory genes relative to a normal control tissue and/or an increase in the methylation of one or more immune checkpoint genes relative to a normal control tissue indicates that the subject should start an anti-cancer regimen that is not an immunotherapy. Also disclosed herein are methods of assessing the suitability of an immunotherapy treatment regimen for the treatment of an immunogenic cancer or metastasis in a subject, wherein the assessment is conducted after to the commencement of an immunotherapy regimen; wherein an increase in the methylation of one or more co-stimulatory genes relative to a normal control tissue and/or a decrease in the methylation of one or more immune checkpoint genes relative to a normal control tissue indicates that the subject can continue an immunotherapy regimen; and wherein a decrease or same amount of methylation of one or more co-stimulatory genes relative to a normal control tissue and/or an increase or same amount of methylation of one or more immune checkpoint genes relative to a normal control tissue indicates that the subject should discontinue an anti-cancer regimen that is not an immunotherapy.

As noted above, methylation, and in particular, hypermethylation (i.e., an increase in methylation relative to a normal tissue control) can lead to a decrease in gene expression of co-stimulatory genes which reduces an immune response to a cancer. Thus, decreasing methylation in an hypermethylated of co-stimulatory genes through, for example, the administration of any inhibitor of methylation (such as, for example, azacytidine, decitabine, and/or zebularine) can alone or in combination with an immunotherapy (including, any of the immune checkpoint inhibitor blockades disclosed herein) decrease, inhibit, ameliorate, reduce, treat, and/or prevent a cancer or metastasis. However, administration of an inhibitor of methylation when co-stimulatory genes are hypomethylated and/or immune checkpoint genes are hypermethylated can have a detrimental effect. Accordingly, disclosed herein are methods of assessing the suitability of an immunotherapy treatment regimen for the treatment of an immunogenic cancer or metastasis in a subject of any preceding aspect, wherein a decrease in the methylation of one or more co-stimulatory genes relative to a normal control tissue or an increase in the methylation of one or more immune checkpoint genes relative to a normal control indicates that an inhibitor of methylation should not be administered to the subject. Also disclosed herein are methods of assessing the suitability of an immunotherapy treatment regimen for the treatment of an immunogenic cancer or metastasis in a subject, wherein an increase in the methylation of one or more co-stimulatory genes relative to a normal control tissue indicates that an inhibitor of methylation can be administered to the subject.

The disclosed treatment methods and assessment methods are suitable for any immunotherapy known to those of skill in the art, including, but not limited to the administration of antibodies, cytokines, natural killer (NK) cells, chimeric antigen receptor (CAR) T cells, CAR NK cells, tumor infiltrating lymphocytes (TILs), marrow infiltrating lymphocytes (MILs), and/or tumor infiltrating NK cells (TINKs) that target or modulate immune response. This includes immune checkpoint inhibitor blockades (such as, for example, antibodies that block PD-1 (Nivolumab (BMS-936558 or MDX1106), CT-011, MK-3475), PD-L1 (MDX-1105 (BMS-936559), Durvalumab, Avelumab, Atezolizumab), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016)). In particular, where hypermethylation (i.e., an increase in methylation relative to a normal tissue control) of a co-stimulatory gene or hypomethylation (i.e., an decrease in methylation relative to a normal tissue control) of an immune checkpoint gene is detected, the treatment methods can include or further include the administration of immunotherapy, including, but not limited to checkpoint inhibitors. Examples of checkpoint inhibitors include, but are not limited to antibodies that block PD-1 (Nivolumab (BMS-936558 or MDX1106), CT-011, MK-3475), PD-L1 (MDX-1105 (BMS-936559), Durvalumab, Avelumab, Atezolizumab), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016). Similarly, disclosed herein are methods of assessing the suitability of an immunotherapy treatment regimen for the treatment of an immunogenic cancer or metastasis in a subject, wherein the immunotherapy comprises an antibody, cytokine, natural killer (NK) cell, chimeric antigen receptor (CAR) T cell, CAR NK cell, tumor infiltrating lymphocyte (TIL), marrow infiltrating lymphocyte (MIL), and/or tumor infiltrating NK cell (TINK)), for example, an immune checkpoint inhibitor blockade.

It is understood and herein contemplated that the assessment of methylation used in the disclosed methods of treating and assessing a treatment regimen can be accomplished by any means known in the art, including, but not limited to principal component analysis, mass spectrometry, High Performance Liquid Chromatography (HPLC), Enzyme-Linked Immunosorbant Assay (ELISA), PCR, bead array, methylation specific PCR, pyrosequencing, bisulfite sequencing, digestion based assay followed by PCR, and/or LUMA. Thus, also disclosed herein are methods of treating, inhibiting, reducing, ameliorating, and/or preventing an immunogenic cancer or metastasis, as well as, methods of assessing the suitability of an immunotherapy treatment regimen for the treatment of an immunogenic cancer or metastasis, wherein methylation is measured by performing principal component (PC) analysis (PCA) of the one or more co-stimulatory genes and/or one or more immune checkpoint genes; wherein PC^{high} indicates an increase in methylation and PC^{low} indicates a decrease in methylation.

The disclosed methods comprise obtaining tissue samples. As used herein, “tissue sample” can comprise any solid or liquid tissue from a subject including, but not limited to biopsy, blood, urine, sputum, saliva, tissue lavage. Tissue samples can be obtained by any means known in the art including but not limited to swab, catch collection, tissue resection, biopsy phlebotomy, and/or core biopsy.

The disclosed methods can be used to treat or assess the treatment of any disease where uncontrolled cellular proliferation occurs such as cancers. A non-limiting list of different types of cancers comprises lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. For example, the cancer can comprise adrenocortical carcinoma, bladder urothelial carcinoma, breast invasive carcinoma, cervical squamous cell carcinoma and endocervical adenocarcinoma, cholangiocarcinoma, colon adenocarcinoma, lymphoid neoplasm diffuse large B-cell lymphoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ sell tumors, thyroid carcinoma, thymoma, uterine corpus endometrial carcinoma, uterine carcinosarcoma, or uveal melanoma.

In one aspect, it is understood and herein contemplated that successful treatment of a cancer in a subject is important and doing so may include the administration of additional treatments. This is particular true where hypermethylation (i.e., an increase in methylation relative to a normal tissue control) of a co-stimulatory gene or hypomethylation (i.e., an decrease in methylation relative to a normal tissue control) of an immune checkpoint gene is not detected as the cancer is less susceptible and/or resistant to immunotherapy (including, but not limited to immune checkpoint inhibitor blockade). Thus, the disclosed treatments can include and/or further include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil--Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride , Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A.R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Example 1: Methylation Modulates the Tumor Immune Synapse

Unprecedented clinical success with immune checkpoint inhibitors alludes to the pivotal importance of the immune synapse that forms between the antigen presenting cells and the effector T-cells. Professional antigen presenting cells such as dendritic cells present tumor-associated antigens via human leukocyte antigen complex (HLA) to the cognate T-cells to elicit tumor-specific immune responses. This high-fidelity recognition of tumor antigen by effector T-cells is either augmented by concomitant interaction of co-stimulatory molecules leading to a functional immune response, or interrupted by engagement of immune checkpoint molecules mediating T-cell anergy or exhaustion.

While professional antigen presenting cells are deemed critical for elicitation of a competent immune response, the immune synapse also forms between the tumor and the effector T-cells; thus, the tumor cells may evade the effector T-cells by neutralizing this interaction. In fact, the interaction between tumor cells and immune cells can shape the immune-suppressive landscape within the tumor microenvironment via mechanisms involved in downregulation of expression of both HLA and a wide array of immune checkpoint and co-stimulatory ligands to modulate T-cell responses. Indeed, the role of tumor in the immune synapse is best illustrated by a tendency of superior efficacy of PD1 blocking antibodies against tumors expressing high levels of PDL1.

Expression of HLA and co-stimulatory/immune checkpoint molecules is intricately modulated at transcription, translation and post-translational levels. In particular, DNA methylation is a crucial epigenetic mechanism of immune regulation with critical roles in T-cell development and differentiation, antigen presentation, effector function and immunologic memory. Because cancer cells frequently utilize epigenetic dysregulation to silence tumor suppressors or activate oncogenes, we hypothesized that tumor progression requires epigenetic reprogramming of immune synapse genes to evade immune killing.

a) Results and Discussions

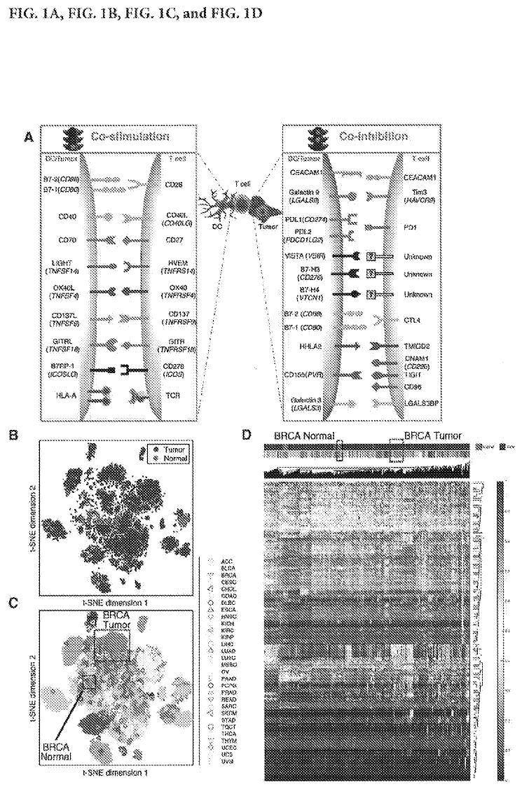

Tumor evolution to evade immune-surveillance is a hallmark of carcinogenesis, and modulation of the immune synapse between antigen presenting cells and effector T-cells directly impacts tumor-specific immunity. As APCs and tumor modulate effector T-cells via ligands for co-stimulatory and immune checkpoint pathways, we focused on the methylation status of these ligands in tumor (FIG. 1A). The Cancer Genome Atlas (TCGA) Level 1 methylation data from 30 solid tumor types were studied (Table 1). Twenty selected genes were divided into two groups, immune checkpoint genes (ICG; i.e., inhibitory) and co-stimulatory genes (CSG; i.e., stimulatory), (Table 2). Of note, CD80 and CD86 have dual roles as both stimulatory when interacting with CD28 or inhibitory as a ligand for CTLA-4. Their affinity is stronger for CTLA-4 and thus likely to mediate inhibitory signals when expressed in low levels, as is generally the case in tumors. Therefore, these two genes were categorized as inhibitory genes in the tumor-immune synapse.

TABLE 1
List of 30 TCGA tumor types

| TCGA_ID | Description |
|---|---|
| ACC | Adrenocortical carcinoma |
| BLCA | Bladder Urothelial Carcinoma |
| BRCA | Breast invasive carcinoma |
| CESC | Cervical squamous cell carcinoma |
| | and endocervical adenocarcinoma |
| CHOL | Cholangiocarcinoma |
| COAD | Colon adenocarcinoma |
| DLBC | Lymphoid Neoplasm Diffuse |
| | Large B-cell Lymphoma |
| ESCA | Esophageal carcinoma |
| HNSC | Head and Neck squamous cell carcinoma |
| KICH | Kidney Chromophobe |
| KIRC | Kidney renal clear cell carcinoma |
| KIRP | Kidney renal papillary cell carcinoma |
| LIHC | Liver hepatocellular carcinoma |
| LUAD | Lung adenocarcinoma |
| LUSC | Lung squamous cell carcinoma |
| MESO | Mesothelioma |
| OV | Ovarian serous cystadenocarcinoma |
| PAAD | Pancreatic adenocarcinoma |
| PCPG | Pheochromocytoma and Paraganglioma |
| PRAD | Prostate adenocarcinoma |
| READ | Rectum adenocarcinoma |
| SARC | Sarcoma |
| SKCM | Skin Cutaneous Melanoma |
| STAD | Stomach adenocarcinoma |
| TGCT | Testicular Germ Cell Tumors |
| THCA | Thyroid carcinoma |
| THYM | Thymoma |
| UCEC | Uterine Corpus Endometrial Carcinoma |
| UCS | Uterine Carcinosarcoma |
| UVM | Uveal Melanoma |

TABLE 2
List of all Immune synapse genes

| | | Gene |
|---|---|---|
| Alternative name | Type | Symbol |
| CEACAM1 | Inhibitory | CEACAM1 |
| Galectin 9 | Inhibitory | LGALS9 |
| PDL1 | Inhibitory | CD274 |
| PDL2 | Inhibitory | PDCD1LG2 |
| VISTA | Inhibitory | C10orf54 |
| B7-H3 | Inhibitory | CD276 |
| B7-H4 | Inhibitory | VTCN1 |
| B7-2 (CD86) | Inhibitory | CD86 |
| B7-1 (CD80) | Inhibitory | CD80 |
| HHLA2 | Inhibitory | HHLA2 |
| CD155 | Inhibitory | PVR |
| Galectin 3 | Inhibitory | LGALS3 |
| CD40 | Stimulatory | CD40 |
| CD70 | Stimulatory | CD70 |
| LIGHT | Stimulatory | TNFSF14 |
| OX40L | Stimulatory | TNFSF4 |
| CD137L (4-1BBL) | Stimulatory | TNFSF9 |
| GITRL | Stimulatory | TNFSF18 |
| B7RP1 | Stimulatory | ICOSLG |
| HLA-A | Stimulatory | HLA-A |

We first investigated whether distinct tumor types were identifiable based on the methylation status of the immune synapse genes using two dimensional t-distributed stochastic neighbor embedding (t-SNE) and unbiased hierarchical clustering analysis. Strikingly, patients with the same tumor type clustered together regardless of other clinical characteristics including age, sex or stage (FIG. 1B-D). This finding indicates the methylation status of immune synapse genes is heavily imprinted by the tissue of origin. By contrast, normal adjacent tissue of the same histology differentially segregated within the cluster highlighting the epigenetic evolution of tumors during carcinogenesis (FIG. 1B-D). For instance, breast cancer (inverted pink triangle) is clearly separated from its counterpart normal adjacent tissue.

Unbiased t-SNE and hierarchical clustering analysis demonstrated that the methylation status of immune synapse genes alone can distinguish tumor vs. normal tissue and histologic subtypes opening up an intriguing possibility that the methylation status of immune synapse genes can be utilized for early detection of cancer.

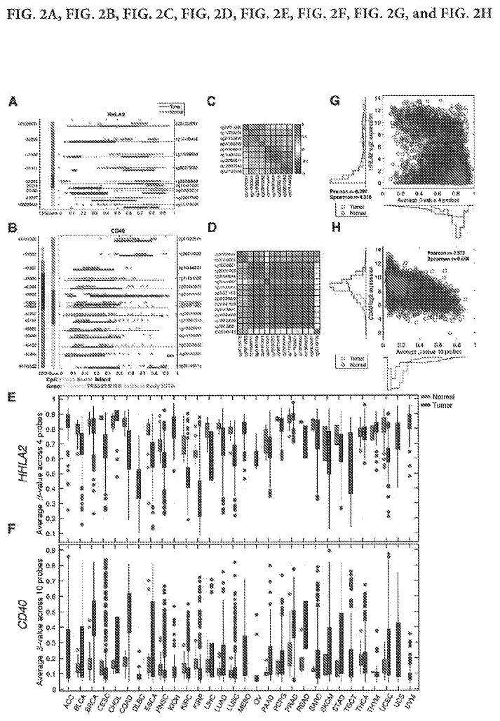

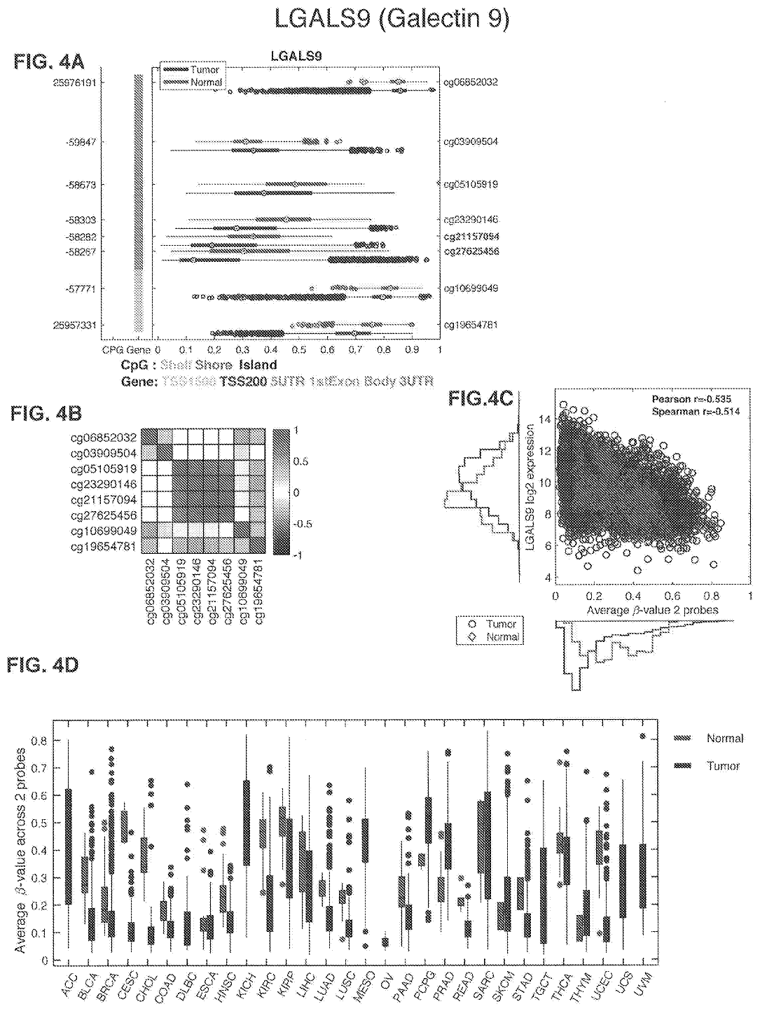

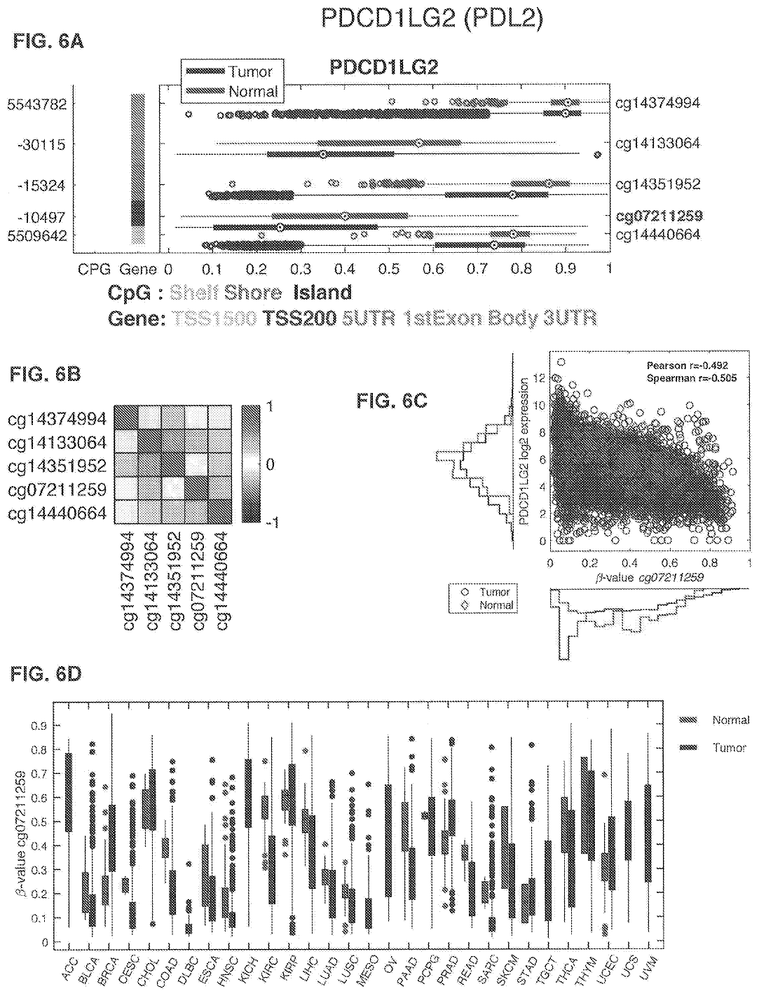

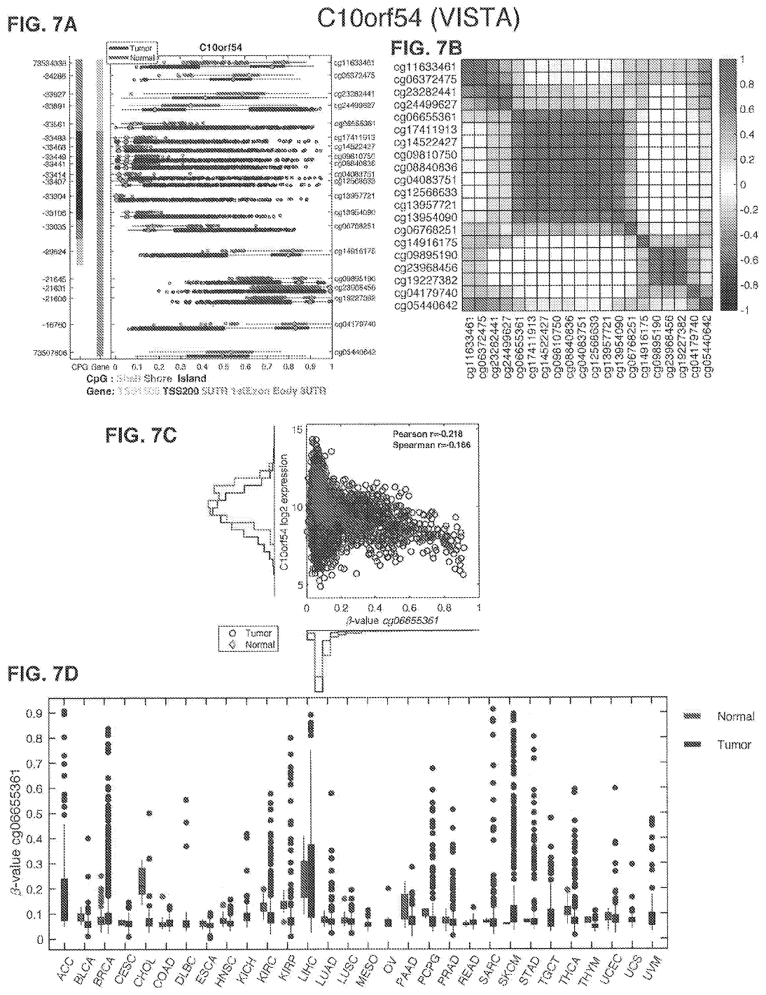

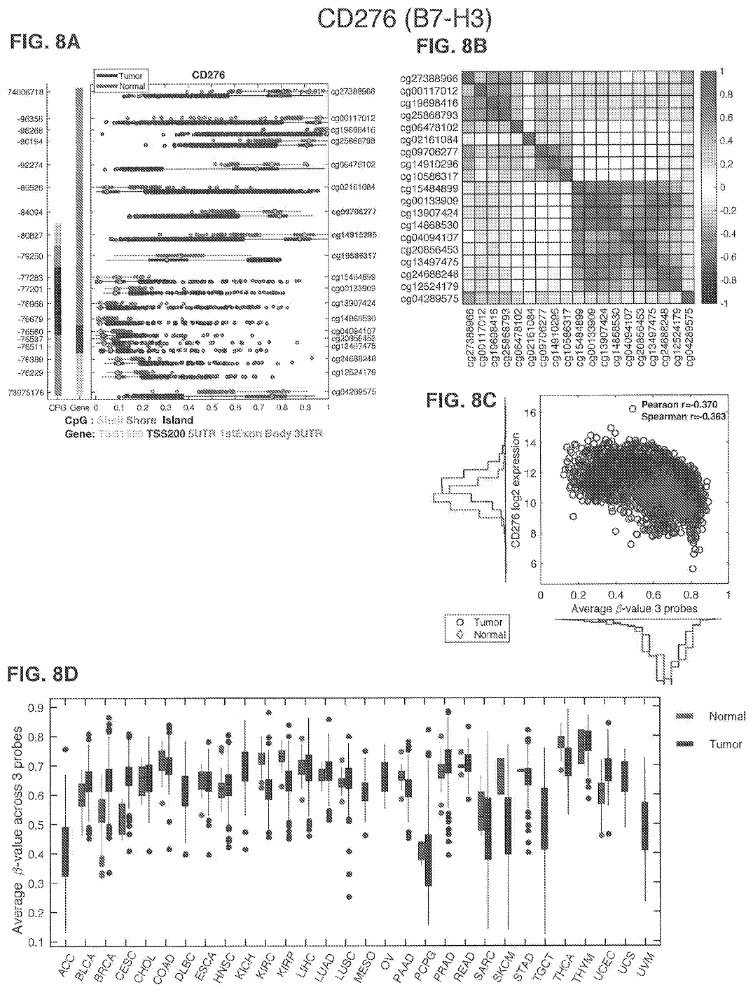

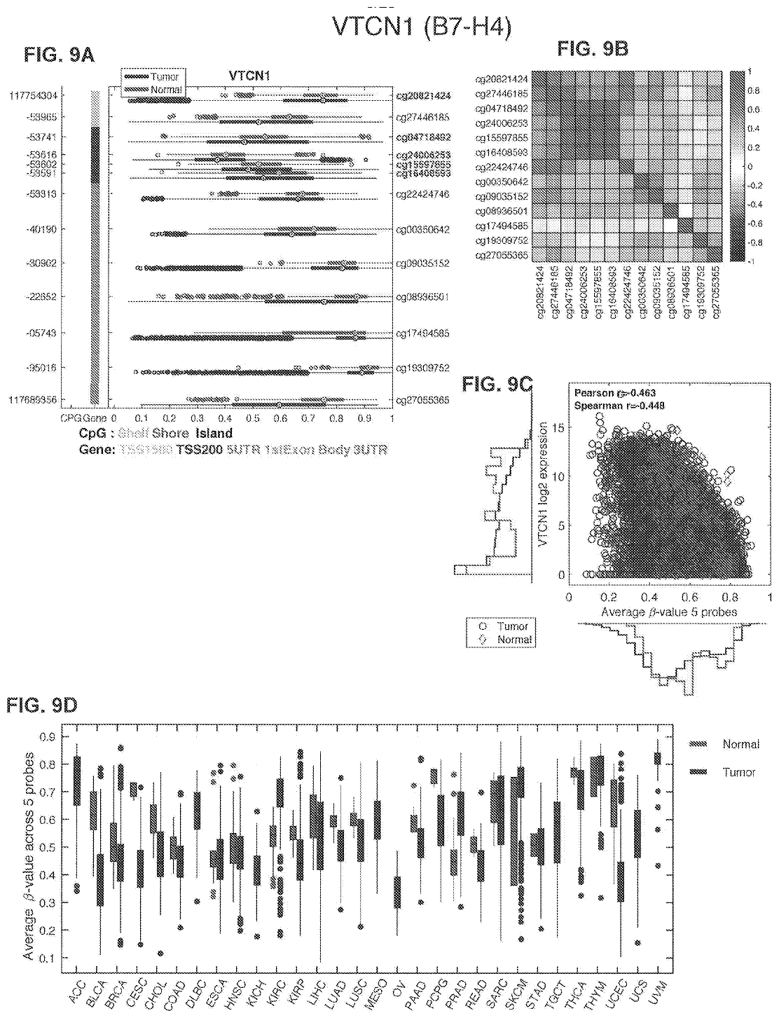

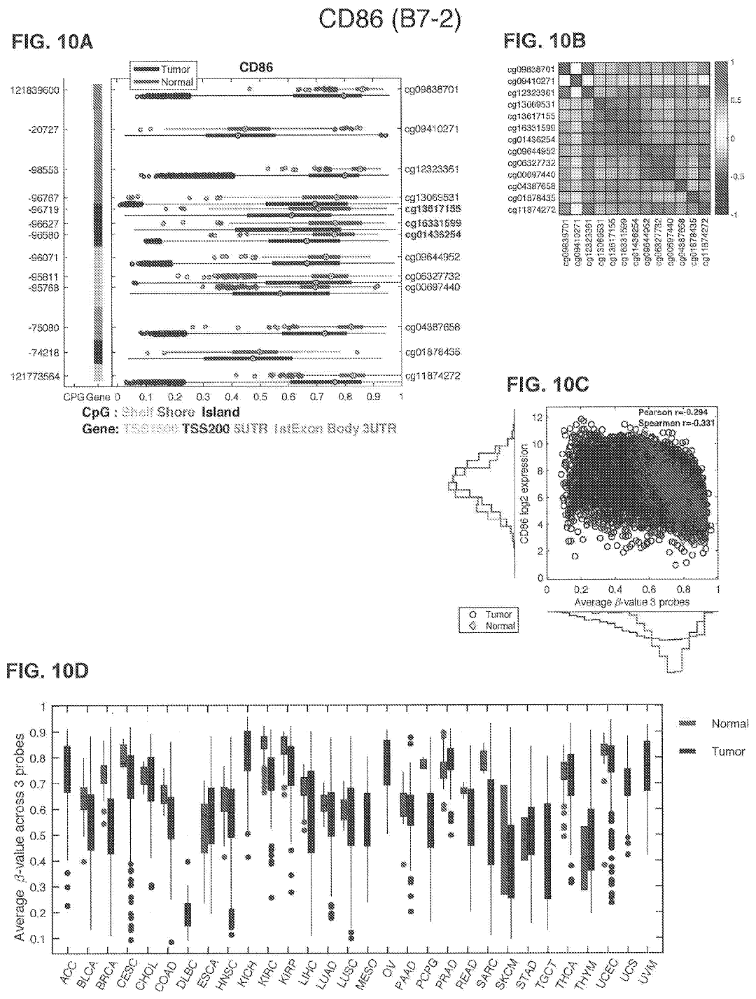

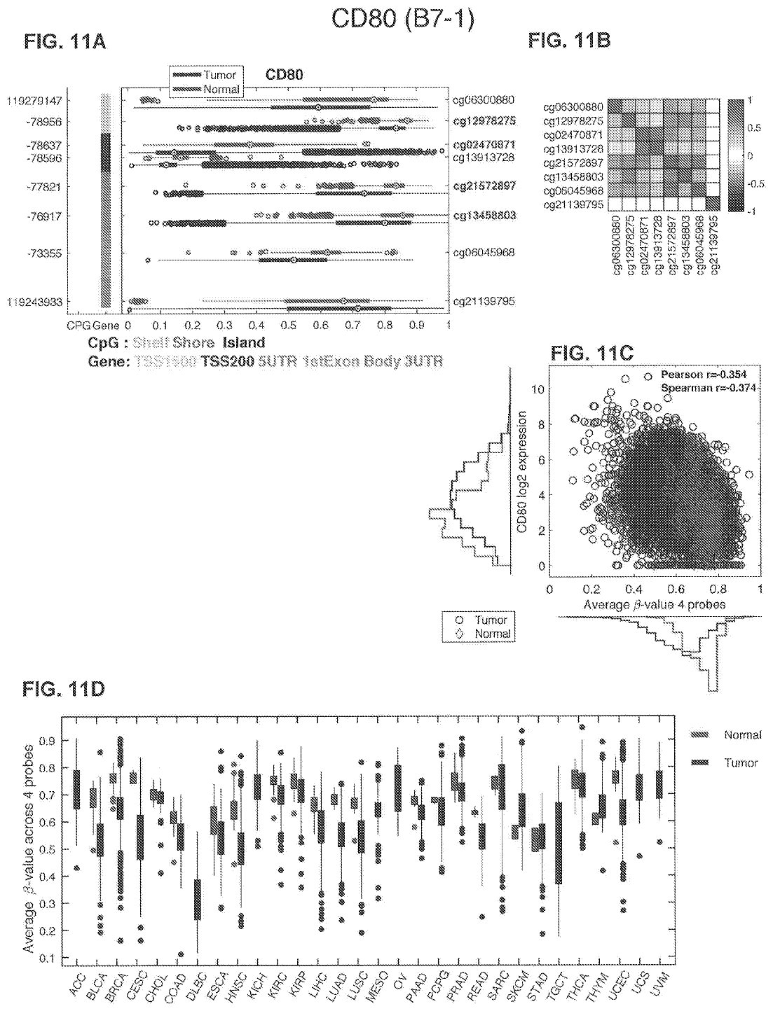

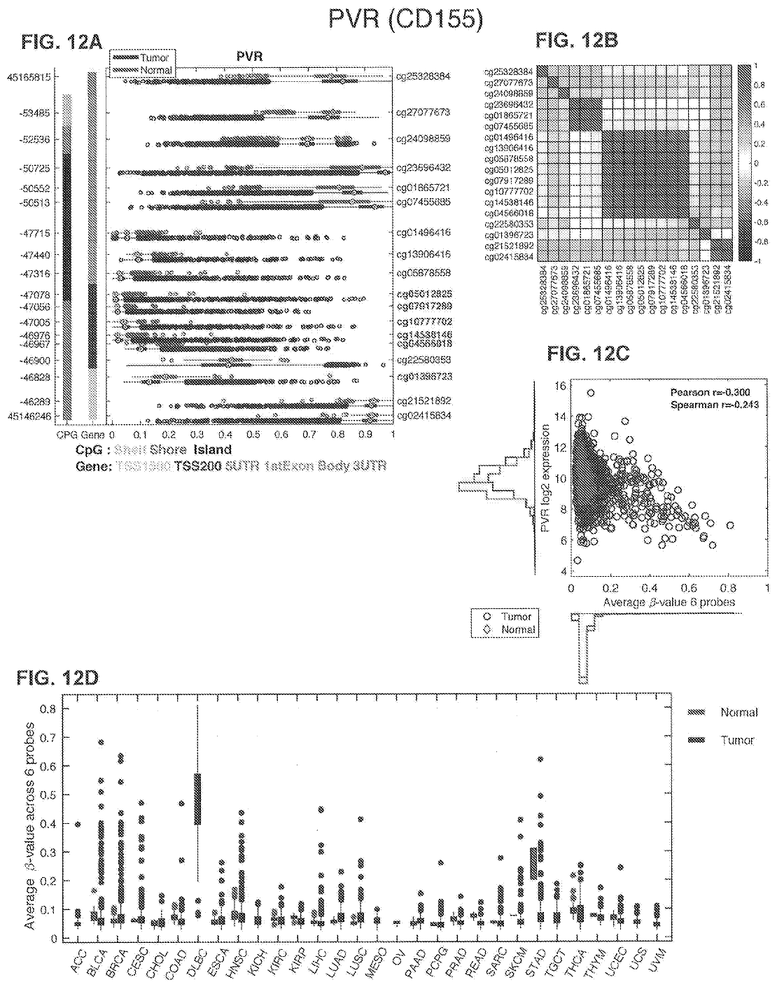

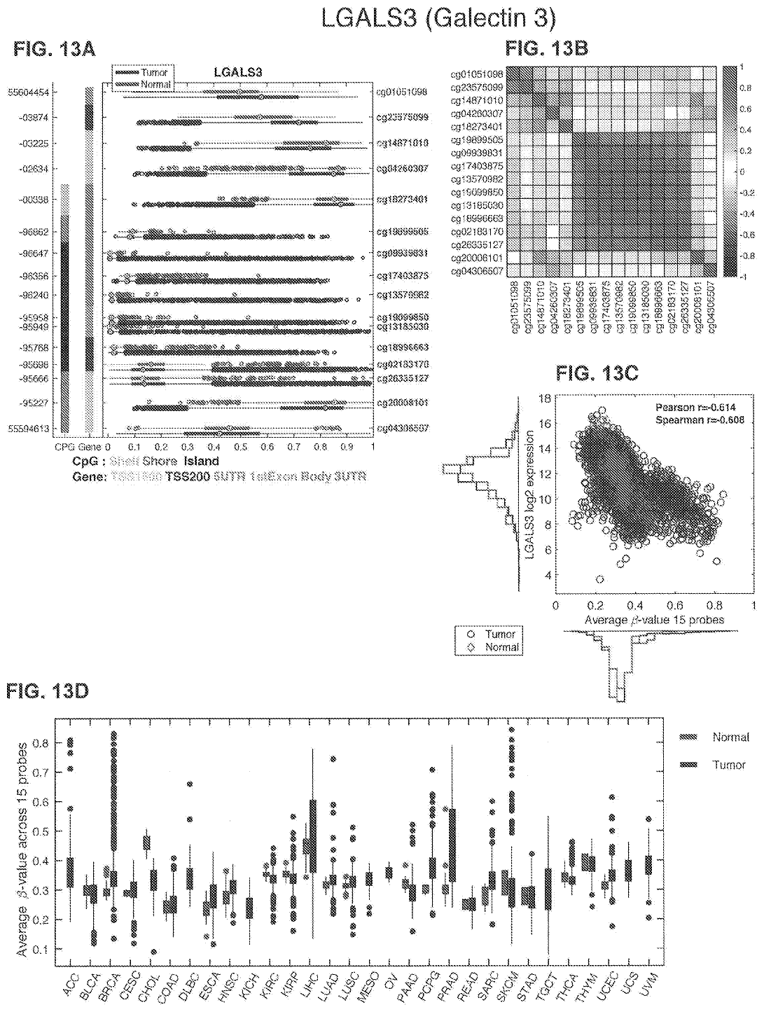

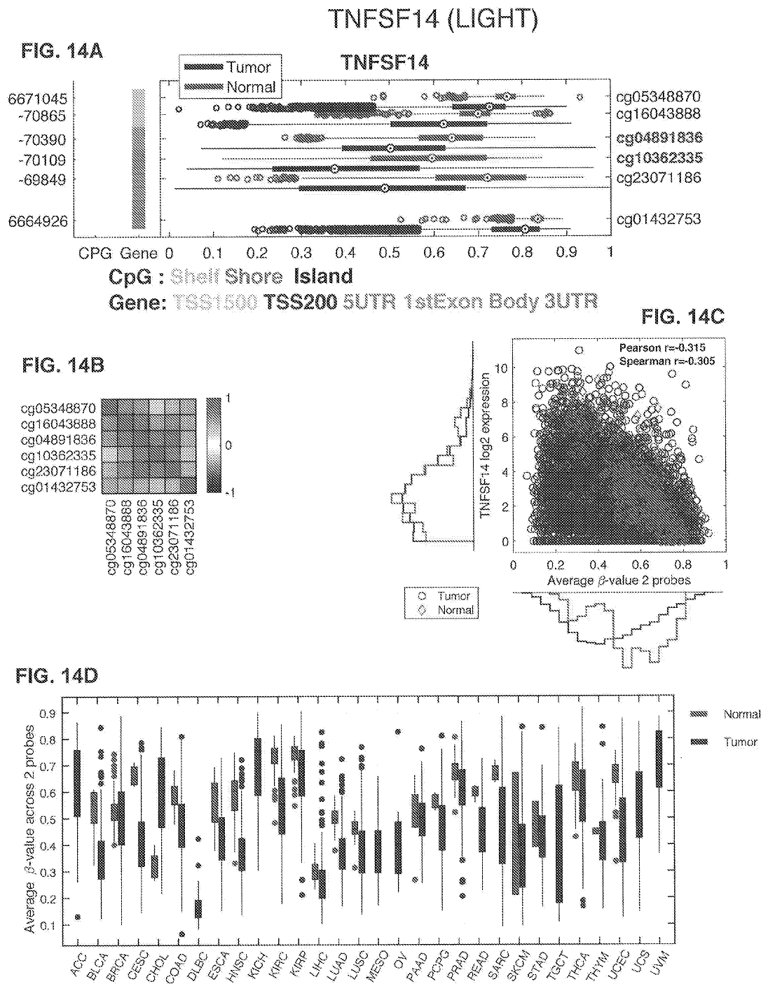

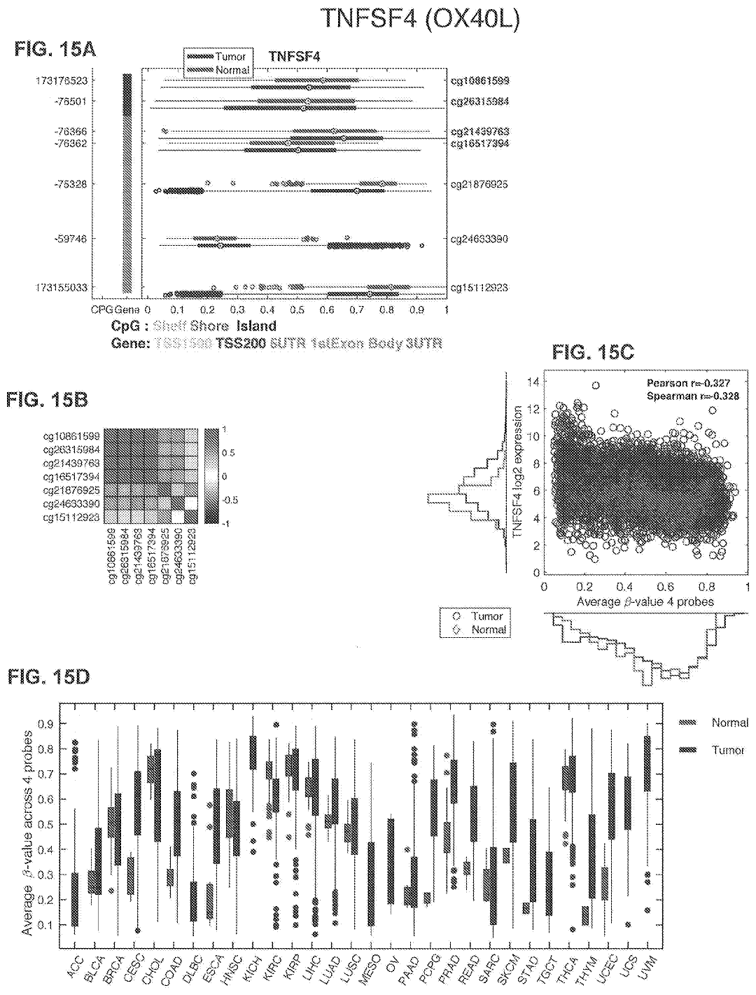

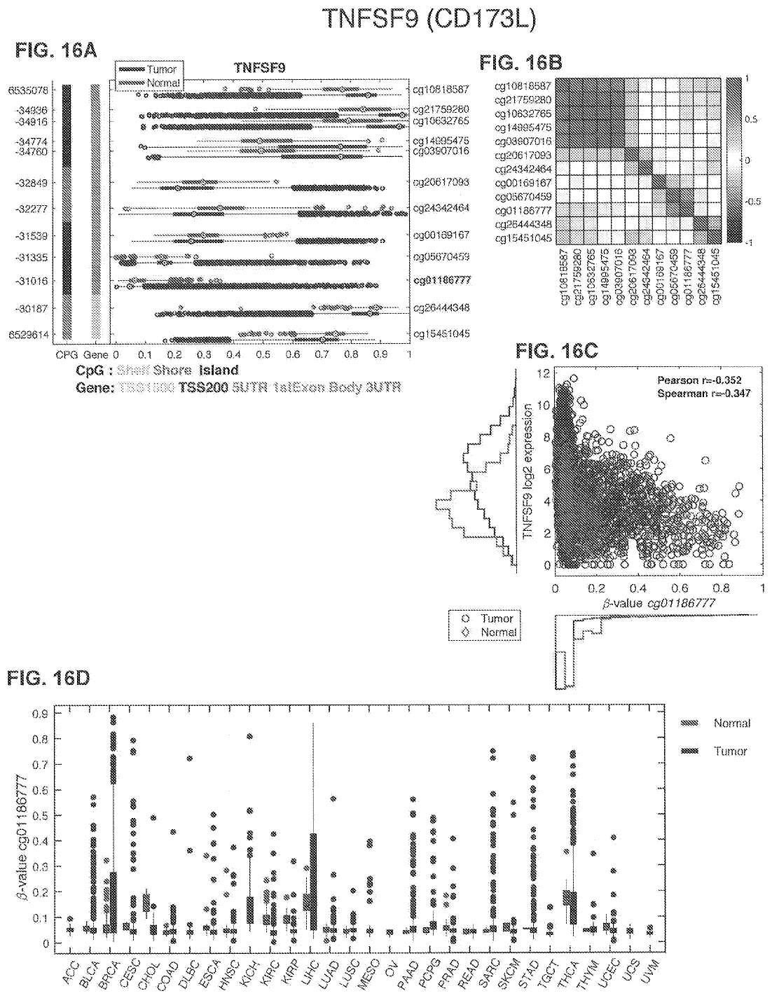

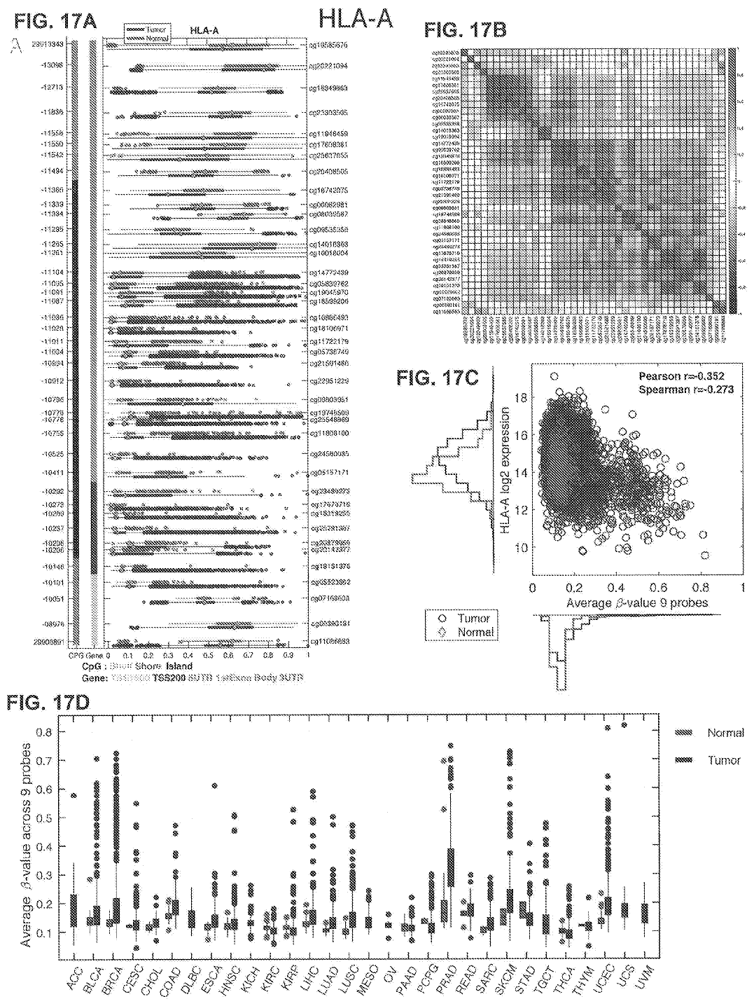

Next, we endeavored to understand the biologic basis of separation between the tumor and the normal adjacent tissue by the methylation status of ICG and CSG by analyzing the methylation pattern of individual genes and their CpG-probes on the 450K chip. A full list of the genes and their probes is given in Table 3. Recent studies have demonstrated that DNA methylation of gene bodies also contribute to transcriptional regulation, however, the probes targeting the putative promoter region of the genes within TSS1500, TSS200, and 5′UTR were evaluated. Interestingly, ICGs and CSGs demonstrated inverse methylation patterns reflecting their opposite immunomodulatory functions (FIG. 2-17). For instance, the β-values of probes within the CD40 gene locus, a prominent CSG, have demonstrated profound hypermethylation in the tumor while the HHLA2 gene locus, an ICG, demonstrated hypomethylation in the tumor in comparison to the normal adjacent tissue (FIG. 2A-B). By contrast, the opposite phenomenon was observed for the CSG genes with an increased methylation in tumor vs. normal adjacent tissue. The correlation between probes within the same gene is high, indicating the consistence of the methylation level measurements (FIG. 2C). Similarly, the CD40 gene locus demonstrated a concordant methylation status with the exception of two probes located in the body and 3′UTR regions of the gene unlikely to be involved in transcriptional control of the gene (FIG. 2D). The average methylation level was calculated using probes located in the TSS1500, TSS200 or 5′UTR region of the gene and with a r<−0.2 (Table 3). Further, the average β-value of the selected probes within the HHLA2 and CD40 gene loci demonstrated consistent methylation patterns across disease sites (FIG. 2E-F): hypermethylation of CD40 and hypomethylation of HHLA2 in comparison to the normal adjacent tissue. Additionally, for both HHLA2 and CD40, the tumor samples demonstrated a larger variance in the methylation levels in tumor vs. normal tissue across disease sites (FIG. 2E-F). Because the known epigenetic mechanism of gene methylation is transcriptional suppression, we interrogated the relationship between the methylation status and its gene expression. As anticipated, an inverse correlation between methylation and gene expression was manifest among tumor and normal adjacent tissue (FIG. 2G-H). Such inverse relationship however was confined to tumor samples with detectable gene expression (i.e. log2 expression>4) (FIG. 2G).

TABLE 3
List of Illumina 450 probes for immune synapse genes.

| | | | | | In 75 | | | | | | |
|---|---|---|---|---|---|---|---|---|---|---|---|
| | Alternative | | | | selected | | | | | | |
| # | name | Type | Num | Gene Symbol | probes | ProbeId | Chr | ChrPos | Islandtype | GeneGroup | r Pearson |
| 1 | CEACAM1 | Inhibitory | 1 | CEACAM1; | No | cg08174715 | 19 | 43012255 | | 3′UTR; | 0.218935 |
| | | | | CEACAM1 | | | | | | 3′UTR | |
| 2 | CEACAM1 | Inhibitory | 2 | CEACAM1; | Yes | cg14904363 | 19 | 43032587 | | 5′UTR; | −0.416157 |
| | | | | CEACAM1; | | | | | | 1stExon; | |
| | | | | CEACAM1; | | | | | | 1stExon; | |
| | | | | CEACAM1 | | | | | | 5′UTR | |
| 3 | CEACAM1 | Inhibitory | 3 | CEACAM1; | Yes | cg11811510 | 19 | 43032683 | | TSS200; | −0.58287 |
| | | | | CEACAM1 | | | | | | TSS200 | |
| 4 | CEACAM1 | Inhibitory | 4 | CEACAM1; | Yes | cg20657383 | 19 | 43033362 | | TSS1500; | −0.374552 |
| | | | | CEACAM1 | | | | | | TSS1500 | |
| 5 | CEACAM1 | Inhibitory | 5 | CEACAM1; | No | cg19776453 | 19 | 43033801 | | TSS1500; | −0.196006 |
| | | | | CEACAM1 | | | | | | TSS1500 | |
| 6 | Galectin 9 | Inhibitory | 1 | LGALS9; | No | cg19654781 | 17 | 25957331 | | TSS1500; | −0.114948 |
| | | | | LGALS9; | | | | | | TSS1500; | |
| | | | | LGALS9 | | | | | | TSS1500 | |
| 7 | Galectin 9 | Inhibitory | 2 | LGALS9; | No | cg10699049 | 17 | 25957771 | | TSS1500; | 0.0411219 |
| | | | | LGALS9; | | | | | | TSS1500; | |
| | | | | LGALS9 | | | | | | TSS1500 | |
| 8 | Galectin 9 | Inhibitory | 3 | LGALS9; | Yes | cg27625456 | 17 | 25958267 | | Body; | −0.501265 |
| | | | | LGALS9; | | | | | | 1stExon; | |
| | | | | LGALS9; | | | | | | 1stExon; | |
| | | | | LGALS9; | | | | | | 5′UTR; | |
| | | | | LGALS9 | | | | | | 5′UTR | |
| 9 | Galectin 9 | Inhibitory | 4 | LGALS9; | Yes | cg21157094 | 17 | 25958282 | | Body; | −0.542128 |
| | | | | LGALS9; | | | | | | 1stExon; | |
| | | | | LGALS9; | | | | | | 1stExon; | |
| | | | | LGALS9; | | | | | | 5′UTR; | |
| | | | | LGALS9 | | | | | | 5′UTR | |
| 10 | Galectin 9 | Inhibitory | 5 | LGALS9; | No | cg23290146 | 17 | 25958303 | | Body; | −0.507165 |
| | | | | LGALS9; | | | | | | 1stExon; | |
| | | | | LGALS9 | | | | | | 1stExon | |
| 11 | Galectin 9 | Inhibitory | 6 | LGALS9; | No | cg05105919 | 17 | 25958673 | | Body; | −0.522717 |
| | | | | LGALS9; | | | | | | Body; | |
| | | | | LGALS9 | | | | | | Body | |
| 12 | Galectin 9 | Inhibitory | 7 | LGALS9; | No | cg03909504 | 17 | 25959847 | | Body; | 0.0871213 |
| | | | | LGALS9; | | | | | | Body; | |
| | | | | LGALS9 | | | | | | Body | |
| 13 | Galectin 9 | Inhibitory | 8 | LGALS9; | No | cg06852032 | 17 | 25976191 | | Body; | 0.116474 |
| | | | | LGALS9; | | | | | | 3′UTR; | |
| | | | | LGALS9 | | | | | | 3′UTR | |
| 14 | PDL1 | Inhibitory | 1 | CD274 | Yes | cg15837913 | 9 | 5449890 | N_Shore | TSS1500 | −0.218126 |
| 15 | PDL1 | Inhibitory | 2 | CD274 | No | cg02823866 | 9 | 5450410 | Island | TSS200 | 0.0544956 |
| 16 | PDL1 | Inhibitory | 3 | CD274 | No | cg14305799 | 9 | 5450535 | Island | TSS200 | 0.00970366 |
| 17 | PDL1 | Inhibitory | 4 | CD274 | No | cg13474877 | 9 | 5450724 | S_Shore | 5′UTR | −0.122665 |
| 18 | PDL1 | Inhibitory | 5 | CD274 | Yes | cg19724470 | 9 | 5450936 | S_Shore | 5′UTR | −0.378707 |
| 19 | PDL2 | Inhibitory | 1 | PDCD1LG2 | No | cg14440664 | 9 | 5509642 | | TSS1500 | −0.110612 |
| 20 | PDL2 | Inhibitory | 2 | PDCD1LG2 | Yes | cg07211259 | 9 | 5510497 | | TSS200 | −0.492481 |
| 21 | PDL2 | Inhibitory | 3 | PDCD1LG2 | No | cg14351952 | 9 | 5515324 | | 5′UTR | 0.289053 |
| 22 | PDL2 | Inhibitory | 4 | PDCD1LG2 | No | cg14133064 | 9 | 5530115 | | Body | 0.0263889 |
| 23 | PDL2 | Inhibitory | 5 | PDCD1LG2 | No | cg14374994 | 9 | 5543782 | | Body | 0.111178 |
| 24 | VISTA | Inhibitory | 1 | C10orf54; | No | cg05440642 | 10 | 73507806 | | 3′UTR; | −0.331883 |
| | | | | CDH23 | | | | | | Body | |
| 25 | VISTA | Inhibitory | 2 | CDH23; | No | cg04179740 | 10 | 73516760 | | Body; | −0.116387 |
| | | | | C10orf54 | | | | | | Body | |
| 26 | VISTA | Inhibitory | 3 | CDH23; | No | cg19227382 | 10 | 73521606 | | Body; | 0.143279 |
| | | | | C10orf54 | | | | | | Body | |
| 27 | VISTA | Inhibitory | 4 | CDH23; | No | cg23968456 | 10 | 73521631 | | Body; | 0.149368 |
| | | | | C10orf54 | | | | | | Body | |
| 28 | VISTA | Inhibitory | 5 | CDH23; | No | cg09895190 | 10 | 73521645 | | Body; | 0.0618893 |
| | | | | C10orf54 | | | | | | Body | |
| 29 | VISTA | Inhibitory | 6 | CDH23; | No | cg14916175 | 10 | 73529624 | N_Shelf | Body; | −0.168602 |
| | | | | C10orf54 | | | | | | Body | |
| 30 | VISTA | Inhibitory | 7 | CDH23; | No | cg06768251 | 10 | 73533035 | N_Shore | Body; | −0.301248 |
| | | | | C10orf54 | | | | | | Body | |
| 31 | VISTA | Inhibitory | 8 | CDH23; | No | cg13954090 | 10 | 73533106 | Island | Body; | −0.270241 |
| | | | | C10orf54 | | | | | | Body | |
| 32 | VISTA | Inhibitory | 9 | C10orf54; | No | cg13957721 | 10 | 73533304 | Island | 5′UTR; | −0.133001 |
| | | | | C10orf54; | | | | | | 1stExon; | |
| | | | | CDH23 | | | | | | Body | |
| 33 | VISTA | Inhibitory | 10 | CDH23; | No | cg12568633 | 10 | 73533407 | Island | Body; | −0.14949 |
| | | | | C10orf54 | | | | | | TSS200 | |
| 34 | VISTA | Inhibitory | 11 | CDH23; | No | cg04083751 | 10 | 73533414 | Island | Body; | −0.139335 |
| | | | | C10orf54 | | | | | | TSS200 | |
| 35 | VISTA | Inhibitory | 12 | CDH23; | No | cg08840836 | 10 | 73533441 | Island | Body; | −0.189989 |
| | | | | C10orf54 | | | | | | TSS200 | |
| 36 | VISTA | Inhibitory | 13 | CDH23; | No | cg09810750 | 10 | 73533449 | Island | Body; | −0.188456 |
| | | | | C10orf54 | | | | | | TSS200 | |
| 37 | VISTA | Inhibitory | 14 | CDH23; | No | cg14522427 | 10 | 73533468 | Island | Body; | −0.171435 |
| | | | | C10orf54 | | | | | | TSS200 | |
| 38 | VISTA | Inhibitory | 15 | CDH23; | No | cg17411913 | 10 | 73533483 | Island | Body; | −0.177352 |
| | | | | C10orf54 | | | | | | TSS200 | |
| 39 | VISTA | Inhibitory | 16 | C10orf54; | Yes | cg06655361 | 10 | 73533561 | S_Shore | TSS1500; | −0.217879 |
| | | | | CDH23 | | | | | | Body | |
| 40 | VISTA | Inhibitory | 17 | C10orf54; | No | cg24499627 | 10 | 73533891 | S_Shore | TSS1500; | 0.0382118 |
| | | | | CDH23 | | | | | | Body | |
| 41 | VISTA | Inhibitory | 18 | C10orf54; | No | cg23282441 | 10 | 73533927 | S_Shore | TSS1500; | 0.0509555 |
| | | | | CDH23 | | | | | | Body | |
| 42 | VISTA | Inhibitory | 19 | C10orf54; | No | cg06372475 | 10 | 73534286 | S_Shore | TSS1500; | −0.132819 |
| | | | | CDH23 | | | | | | Body | |
| 43 | VISTA | Inhibitory | 20 | C10orf54; | No | cg11633461 | 10 | 73534338 | S_Shore | TSS1500; | −0.089301 |
| | | | | CDH23 | | | | | | Body | |
| 44 | B7-H3 | Inhibitory | 1 | CD276; | No | cg04289575 | 15 | 73975176 | N_Shore | TSS1500; | 0.0521741 |
| | | | | CD276 | | | | | | TSS1500 | |
| 45 | B7-H3 | Inhibitory | 2 | CD276; | No | cg12524179 | 15 | 73976229 | N_Shore | TSS1500; | −0.10806 |
| | | | | CD276 | | | | | | TSS1500 | |
| 46 | B7-H3 | Inhibitory | 3 | CD276; | No | cg24688248 | 15 | 73976389 | N_Shore | TSS1500; | −0.153876 |
| | | | | CD276 | | | | | | TSS1500 | |
| 47 | B7-H3 | Inhibitory | 4 | CD276; | No | cg13497475 | 15 | 73976511 | N_Shore | TSS200; | −0.136537 |
| | | | | CD276 | | | | | | TSS200 | |
| 48 | B7-H3 | Inhibitory | 5 | CD276; | No | cg20856453 | 15 | 73976537 | N_Shore | TSS200; | −0.121453 |
| | | | | CD276 | | | | | | TSS200 | |
| 49 | B7-H3 | Inhibitory | 6 | CD276; | No | cg04094107 | 15 | 73976560 | Island | TSS200; | −0.112064 |
| | | | | CD276 | | | | | | TSS200 | |
| 50 | B7-H3 | Inhibitory | 7 | CD276; | No | cg14868530 | 15 | 73976679 | Island | 5′UTR; | −0.165773 |
| | | | | CD276; | | | | | | 1stExon; | |
| | | | | CD276; | | | | | | 1stExon; | |
| | | | | CD276 | | | | | | 5′UTR | |
| 51 | B7-H3 | Inhibitory | 8 | CD276; | No | cg13907424 | 15 | 73976968 | Island | 5′UTR; | −0.193746 |
| | | | | CD276 | | | | | | 5′UTR | |
| 52 | B7-H3 | Inhibitory | 9 | CD276; | No | cg00133909 | 15 | 73977201 | Island | 5′UTR; | −0.185099 |
| | | | | CD276 | | | | | | 5′UTR | |
| 53 | B7-H3 | Inhibitory | 10 | CD276; | No | cg15484899 | 15 | 73977283 | Island | 5′UTR; | −0.169522 |
| | | | | CD276 | | | | | | 5′UTR | |
| 54 | B7-H3 | Inhibitory | 11 | CD276; | Yes | cg10586317 | 15 | 73979250 | S_Shore | 5′UTR; | −0.336759 |
| | | | | CD276 | | | | | | 5′UTR | |
| 55 | B7-H3 | Inhibitory | 12 | CD276; | Yes | cg14910296 | 15 | 73980827 | S_Shelf | 5′UTR; | −0.272279 |
| | | | | CD276 | | | | | | 5′UTR | |
| 56 | B7-H3 | Inhibitory | 13 | CD276; | Yes | cg09706277 | 15 | 73984094 | | 5′UTR; | −0.288548 |
| | | | | CD276 | | | | | | 5′UTR | |
| 57 | B7-H3 | Inhibitory | 14 | CD276; | No | cg02161084 | 15 | 73989526 | | 5′UTR; | −0.11153 |
| | | | | CD276 | | | | | | 5′UTR | |
| 58 | B7-H3 | Inhibitory | 15 | CD276; | No | cg06478102 | 15 | 73992274 | | Body; | 0.0802598 |
| | | | | CD276 | | | | | | Body | |
| 59 | B7-H3 | Inhibitory | 16 | CD276; | No | cg25868793 | 15 | 73996194 | | Body; | 0.0405871 |
| | | | | CD276 | | | | | | Body | |
| 60 | B7-H3 | Inhibitory | 17 | CD276; | No | cg19698416 | 15 | 73996268 | | Body; | 0.0843344 |
| | | | | CD276 | | | | | | Body | |
| 61 | B7-H3 | Inhibitory | 18 | CD276; | No | cg00117012 | 15 | 73996358 | | Body; | 0.0777926 |
| | | | | CD276 | | | | | | Body | |
| 62 | B7-H3 | Inhibitory | 19 | CD276; | No | cg27388966 | 15 | 74006718 | | 3′UTR; | 0.0909163 |
| | | | | CD276 | | | | | | 3′UTR | |
| 63 | B7-H4 | Inhibitory | 1 | VTCN1 | No | cg27055365 | 1 | 117689356 | | 3′UTR | 0.213829 |
| 64 | B7-H4 | Inhibitory | 2 | VTCN1 | No | cg19309752 | 1 | 117695016 | | Body | 0.21785 |
| 65 | B7-H4 | Inhibitory | 3 | VTCN1 | No | cg17494585 | 1 | 117705743 | | Body | 0.0228648 |
| 66 | B7-H4 | Inhibitory | 4 | VTCN1 | No | cg08936501 | 1 | 117722652 | | Body | −0.236615 |
| 67 | B7-H4 | Inhibitory | 5 | VTCN1 | No | cg09035152 | 1 | 117730902 | | Body | 0.0214917 |
| 68 | B7-H4 | Inhibitory | 6 | VTCN1 | No | cg00350642 | 1 | 117740190 | | Body | 0.139957 |
| 69 | B7-H4 | Inhibitory | 7 | VTCN1 | No | cg22424746 | 1 | 117753313 | | Body | −0.311247 |
| 70 | B7-H4 | Inhibitory | 8 | VTCN1 | Yes | cg16408593 | 1 | 117753591 | | TSS200 | −0.42456 |
| 71 | B7-H4 | Inhibitory | 9 | VTCN1 | Yes | cg15597855 | 1 | 117753602 | | TSS200 | −0.458988 |
| 72 | B7-H4 | Inhibitory | 10 | VTCN1 | Yes | cg24006253 | 1 | 117753616 | | TSS200 | −0.398822 |
| 73 | B7-H4 | Inhibitory | 11 | VTCN1 | Yes | cg04718492 | 1 | 117753741 | | TSS200 | −0.435105 |
| 74 | B7-H4 | Inhibitory | 12 | VTCN1 | No | cg27446185 | 1 | 117753965 | | TSS1500 | −0.198633 |
| 75 | B7-H4 | Inhibitory | 13 | VTCN1 | Yes | cg20821424 | 1 | 117754304 | | TSS1500 | −0.308691 |
| 76 | B7-2 | Inhibitory | 1 | CD86 | No | cg11874272 | 3 | 121773564 | | TSS1500 | 0.0168761 |
| 77 | B7-2 | Inhibitory | 2 | CD86 | No | cg01878435 | 3 | 121774218 | | TSS200 | −0.19246 |
| 78 | B7-2 | Inhibitory | 3 | CD86 | No | cg04387658 | 3 | 121775080 | | Body | 0.0958312 |
| 79 | B7-2 | Inhibitory | 4 | CD86; | No | cg00697440 | 3 | 121795768 | | TSS1500; | 0.0457929 |
| | | | | CD86 | | | | | | Body | |
| 80 | B7-2 | Inhibitory | 5 | CD86; | No | cg06327732 | 3 | 121795811 | | TSS1500; | 0.00947203 |
| | | | | CD86 | | | | | | Body | |
| 81 | B7-2 | Inhibitory | 6 | CD86; | No | cg09644952 | 3 | 121796071 | | TSS1500; | −0.128381 |
| | | | | CD86 | | | | | | Body | |
| 82 | B7-2 | Inhibitory | 7 | CD86; | Yes | cg01436254 | 3 | 121796580 | | TSS200; | −0.264535 |
| | | | | CD86 | | | | | | Body | |
| 83 | B7-2 | Inhibitory | 8 | CD86; | Yes | cg16331599 | 3 | 121796627 | | TSS200; | −0.287409 |
| | | | | CD86 | | | | | | Body | |
| 84 | B7-2 | Inhibitory | 9 | CD86; | Yes | cg13617155 | 3 | 121796719 | | TSS200; | −0.247929 |
| | | | | CD86 | | | | | | Body | |
| 85 | B7-2 | Inhibitory | 10 | CD86; | No | cg13069531 | 3 | 121796767 | | 5′UTR; | −0.194598 |
| | | | | CD86; | | | | | | Body; | |
| | | | | CD86 | | | | | | 1stExon | |
| 86 | B7-2 | Inhibitory | 11 | CD86; | No | cg12323361 | 3 | 121798553 | | 5′UTR; | 0.0256737 |
| | | | | CD86 | | | | | | Body | |
| 87 | B7-2 | Inhibitory | 12 | CD86; | No | cg09410271 | 3 | 121820727 | | Body; | 0.0553987 |
| | | | | CD86 | | | | | | Body | |
| 88 | B7-2 | Inhibitory | 13 | CD86; | No | cg09838701 | 3 | 121839600 | | 3′UTR; | 0.094376 |
| | | | | CD86 | | | | | | 3′UTR | |
| 89 | B7-1 | Inhibitory | 1 | CD80 | No | cg21139795 | 3 | 119243933 | | 3′UTR | −0.012676 |
| 90 | B7-1 | Inhibitory | 2 | CD80 | No | cg06045968 | 3 | 119273355 | | Body | 0.0786748 |
| 91 | B7-1 | Inhibitory | 3 | CD80 | Yes | cg13458803 | 3 | 119276917 | | 5′UTR | −0.368677 |
| 92 | B7-1 | Inhibitory | 4 | CD80 | Yes | cg21572897 | 3 | 119277821 | | 5′UTR | −0.207401 |
| 93 | B7-1 | Inhibitory | 5 | CD80 | No | cg13913728 | 3 | 119278596 | | TSS200 | 0.0524523 |
| 94 | B7-1 | Inhibitory | 6 | CD80 | Yes | cg02470871 | 3 | 119278637 | | TSS200 | −0.213288 |
| 95 | B7-1 | Inhibitory | 7 | CD80 | Yes | cg12978275 | 3 | 119278956 | | TSS1500 | −0.33574 |
| 96 | B7-1 | Inhibitory | 8 | CD80 | No | cg06300880 | 3 | 119279147 | | TSS1500 | 0.0958884 |
| 97 | HHLA2 | Inhibitory | 1 | HHLA2 | Yes | cg02124498 | 3 | 108020451 | | TSS1500 | −0.313458 |
| 98 | HHLA2 | Inhibitory | 2 | HHLA2 | Yes | cg08817540 | 3 | 108020727 | | TSS1500 | −0.272822 |
| 99 | HHLA2 | Inhibitory | 3 | HHLA2 | Yes | cg02059214 | 3 | 108021106 | | TSS1500 | −0.387769 |
| 100 | HHLA2 | Inhibitory | 4 | HHLA2 | Yes | cg10431989 | 3 | 108021214 | | TSS200 | −0.380847 |
| 101 | HHLA2 | Inhibitory | 5 | HHLA2 | No | cg22926869 | 3 | 108021293 | | TSS200 | −0.192687 |
| 102 | HHLA2 | Inhibitory | 6 | HHLA2 | No | cg00915092 | 3 | 108031411 | | 5′UTR | 0.0827543 |
| 103 | HHLA2 | Inhibitory | 7 | HHLA2 | No | cg24769830 | 3 | 108041508 | | 5′UTR | 0.0668664 |
| 104 | HHLA2 | Inhibitory | 8 | HHLA2 | No | cg14703454 | 3 | 108065259 | | 5′UTR | 0.165865 |
| 105 | HHLA2 | Inhibitory | 9 | HHLA2 | No | cg27229097 | 3 | 108096637 | | 3′UTR | 0.254071 |
| 106 | CD155 | Inhibitory | 1 | PVR; | No | cg02415834 | 19 | 45146246 | N_Shore | TSS1500; | 0.108791 |
| | | | | PVR; | | | | | | TSS1500; | |
| | | | | PVR; | | | | | | TSS1500; | |
| | | | | PVR | | | | | | TSS1500 | |
| 107 | CD155 | Inhibitory | 2 | PVR; | No | cg21521892 | 19 | 45146289 | N_Shore | TSS1500; | 0.0640571 |
| | | | | PVR; | | | | | | TSS1500; | |
| | | | | PVR; | | | | | | TSS1500; | |
| | | | | PVR | | | | | | TSS1500 | |
| 108 | CD155 | Inhibitory | 3 | PVR; | Yes | cg01396723 | 19 | 45146828 | N_Shore | TSS1500; | −0.274669 |
| | | | | PVR; | | | | | | TSS1500; | |
| | | | | PVR; | | | | | | TSS1500; | |
| | | | | PVR | | | | | | TSS1500 | |
| 109 | CD155 | Inhibitory | 4 | PVR; | No | cg22580353 | 19 | 45146900 | N_Shore | TSS200; | 0.0505606 |
| | | | | PVR; | | | | | | TSS200; | |
| | | | | PVR; | | | | | | TSS200; | |
| | | | | PVR | | | | | | TSS200 | |
| 110 | CD155 | Inhibitory | 5 | PVR; | Yes | cg04566018 | 19 | 45146967 | N_Shore | TSS200; | −0.267189 |
| | | | | PVR; | | | | | | TSS200; | |
| | | | | PVR; | | | | | | TSS200; | |
| | | | | PVR | | | | | | TSS200 | |
| 111 | CD155 | Inhibitory | 6 | PVR; | Yes | cg14538146 | 19 | 45146976 | N_Shore | TSS200; | −0.232072 |
| | | | | PVR; | | | | | | TSS200; | |
| | | | | PVR; | | | | | | TSS200; | |
| | | | | PVR | | | | | | TSS200 | |
| 112 | CD155 | Inhibitory | 7 | PVR; | Yes | cg10777702 | 19 | 45147005 | N_Shore | TSS200; | −0.247643 |
| | | | | PVR; | | | | | | TSS200; | |
| | | | | PVR; | | | | | | TSS200; | |
| | | | | PVR | | | | | | TSS200 | |
| 113 | CD155 | Inhibitory | 8 | PVR; | Yes | cg07917289 | 19 | 45147056 | N_Shore | TSS200; | −0.229176 |
| | | | | PVR; | | | | | | TSS200; | |
| | | | | PVR; | | | | | | TSS200; | |
| | | | | PVR | | | | | | TSS200 | |
| 114 | CD155 | Inhibitory | 9 | PVR; | Yes | cg05012825 | 19 | 45147078 | Island | TSS200; | −0.225405 |
| | | | | PVR; | | | | | | TSS200; | |
| | | | | PVR; | | | | | | TSS200; | |
| | | | | PVR | | | | | | TSS200 | |
| 115 | CD155 | Inhibitory | 10 | PVR; | No | cg05878558 | 19 | 45147316 | Island | 1stExon; | −0.195013 |
| | | | | PVR; | | | | | | 5′UTR; | |
| | | | | PVR; | | | | | | 5′UTR; | |
| | | | | PVR; | | | | | | 1stExon; | |
| | | | | PVR; | | | | | | 5′UTR; | |
| | | | | PVR; | | | | | | 1stExon; | |
| | | | | PVR; | | | | | | 1stExon; | |
| | | | | PVR | | | | | | 5′UTR | |
| 116 | CD155 | Inhibitory | 11 | PVR; | No | cg13906416 | 19 | 45147440 | Island | 1stExon; | −0.175206 |
| | | | | PVR; | | | | | | 1stExon; | |
| | | | | PVR; | | | | | | 1stExon; | |
| | | | | PVR | | | | | | 1stExon | |
| 117 | CD155 | Inhibitory | 12 | PVR; | No | cg01496416 | 19 | 45147715 | Island | Body; | −0.203545 |
| | | | | PVR; | | | | | | Body; | |
| | | | | PVR; | | | | | | Body; | |
| | | | | PVR | | | | | | Body | |
| 118 | CD155 | Inhibitory | 13 | PVR; | No | cg07455685 | 19 | 45150513 | Island | Body; | 0.0921031 |
| | | | | PVR; | | | | | | Body; | |
| | | | | PVR; | | | | | | Body; | |
| | | | | PVR | | | | | | Body | |
| 119 | CD155 | Inhibitory | 14 | PVR; | No | cg01865721 | 19 | 45150552 | Island | Body; | 0.0166044 |
| | | | | PVR; | | | | | | Body; | |
| | | | | PVR; | | | | | | Body; | |
| | | | | PVR | | | | | | Body | |
| 120 | CD155 | Inhibitory | 15 | PVR; | No | cg23696432 | 19 | 45150725 | Island | Body; | 0.0350335 |
| | | | | PVR; | | | | | | Body; | |
| | | | | PVR; | | | | | | Body; | |
| | | | | PVR | | | | | | Body | |
| 121 | CD155 | Inhibitory | 16 | PVR; | No | cg24098859 | 19 | 45152536 | S_Shore | Body; | −0.211099 |
| | | | | PVR; | | | | | | Body; | |
| | | | | PVR; | | | | | | Body; | |
| | | | | PVR | | | | | | Body | |
| 122 | CD155 | Inhibitory | 17 | PVR; | No | cg27077673 | 19 | 45153485 | S_Shelf | Body; | 0.0260456 |
| | | | | PVR; | | | | | | Body; | |
| | | | | PVR; | | | | | | Body; | |
| | | | | PVR | | | | | | Body | |
| 123 | CD155 | Inhibitory | 18 | PVR; | No | cg25328384 | 19 | 45165815 | | 3′UTR; | 0.0426888 |
| | | | | PVR; | | | | | | 3′UTR; | |
| | | | | PVR | | | | | | 3′UTR | |
| 124 | Galectin 3 | Inhibitory | 1 | LGALS3 | Yes | cg04306507 | 14 | 55594613 | N_Shore | TSS1500 | −0.265989 |
| 125 | Galectin 3 | Inhibitory | 2 | LGALS3 | Yes | cg20008101 | 14 | 55595227 | N_Shore | TSS1500 | −0.421086 |
| 126 | Galectin 3 | Inhibitory | 3 | LGALS3 | Yes | cg26335127 | 14 | 55595666 | N_Shore | TSS1500 | −0.532083 |
| 127 | Galectin 3 | Inhibitory | 4 | LGALS3 | Yes | cg02183170 | 14 | 55595698 | Island | TSS200 | −0.613505 |
| 128 | Galectin 3 | Inhibitory | 5 | LGALS3 | Yes | cg18996663 | 14 | 55595768 | Island | TSS200 | −0.462405 |
| 129 | Galectin 3 | Inhibitory | 6 | LGALS3; | Yes | cg13185030 | 14 | 55595949 | Island | 1stExon; | −0.369518 |
| | | | | LGALS3 | | | | | | 5′UTR | |
| 130 | Galectin 3 | Inhibitory | 7 | LGALS3; | Yes | cg19099850 | 14 | 55595958 | Island | 1stExon; | −0.378208 |
| | | | | LGALS3 | | | | | | 5′UTR | |
| 131 | Galectin 3 | Inhibitory | 8 | LGALS3 | Yes | cg13570982 | 14 | 55596240 | Island | 5′UTR | −0.368171 |
| 132 | Galectin 3 | Inhibitory | 9 | LGALS3 | Yes | cg17403875 | 14 | 55596356 | Island | 5′UTR | −0.407798 |
| 133 | Galectin 3 | Inhibitory | 10 | LGALS3 | Yes | cg09939831 | 14 | 55596647 | Island | 5′UTR | −0.338851 |
| 134 | Galectin 3 | Inhibitory | 11 | LGALS3 | Yes | cg19899505 | 14 | 55596862 | S_Shore | 5′UTR | −0.366875 |
| 135 | Galectin 3 | Inhibitory | 12 | LGALS3 | Yes | cg18273401 | 14 | 55600338 | S_Shelf | 5′UTR | −0.23157 |
| 136 | Galectin 3 | Inhibitory | 13 | LGALS3; | Yes | cg04260307 | 14 | 55602634 | | TSS1500; | −0.217037 |
| | | | | LGALS3 | | | | | | 5′UTR | |
| 137 | Galectin 3 | Inhibitory | 14 | LGALS3; | Yes | cg14871010 | 14 | 55603225 | | TSS1500; | −0.365055 |
| | | | | LGALS3 | | | | | | 5′UTR | |
| 138 | Galectin 3 | Inhibitory | 15 | LGALS3; | Yes | cg23575099 | 14 | 55603874 | | TSS200; | −0.275906 |
| | | | | LGALS3 | | | | | | 5′UTR | |
| 139 | Galectin 3 | Inhibitory | 16 | LGALS3; | No | cg01051098 | 14 | 55604454 | | Body; | −0.294323 |
| | | | | LGALS3 | | | | | | Body | |
| 140 | CD40 | Stimulatory | 1 | CD40; | No | cg01149415 | 20 | 44745522 | N_Shore | TSS1500; | 0.22303 |
| | | | | CD40 | | | | | | TSS1500 | |
| 141 | CD40 | Stimulatory | 2 | CD40; | Yes | cg09053081 | 20 | 44746392 | N_Shore | TSS1500; | −0.538294 |
| | | | | CD40 | | | | | | TSS1500 | |
| 142 | CD40 | Stimulatory | 3 | CD40; | Yes | cg19839655 | 20 | 44746499 | N_Shore | TSS1500; | −0.525082 |
| | | | | CD40 | | | | | | TSS1500 | |
| 143 | CD40 | Stimulatory | 4 | CD40; | Yes | cg19785066 | 20 | 44746655 | N_Shore | TSS1500; | −0.560293 |
| | | | | CD40 | | | | | | TSS1500 | |
| 144 | CD40 | Stimulatory | 5 | CD40; | Yes | cg17929951 | 20 | 44746681 | N_Shore | TSS1500; | −0.545697 |
| | | | | CD40 | | | | | | TSS1500 | |
| 145 | CD40 | Stimulatory | 6 | CD40; | Yes | cg11841529 | 20 | 44746751 | N_Shore | TSS200; | −0.527903 |
| | | | | CD40 | | | | | | TSS200 | |
| 146 | CD40 | Stimulatory | 7 | CD40; | Yes | cg25239996 | 20 | 44746767 | N_Shore | TSS200; | −0.501244 |
| | | | | CD40 | | | | | | TSS200 | |
| 147 | CD40 | Stimulatory | 8 | CD40; | Yes | cg06571407 | 20 | 44746823 | Island | TSS200; | −0.541914 |
| | | | | CD40 | | | | | | TSS200 | |
| 148 | CD40 | Stimulatory | 9 | CD40; | Yes | cg22232207 | 20 | 44746825 | Island | TSS200; | −0.540844 |
| | | | | CD40 | | | | | | TSS200 | |
| 149 | CD40 | Stimulatory | 10 | CD40; | Yes | cg24575067 | 20 | 44746902 | Island | TSS200; | −0.225347 |
| | | | | CD40 | | | | | | TSS200 | |
| 150 | CD40 | Stimulatory | 11 | CD40; | Yes | cg01943874 | 20 | 44746944 | Island | 1stExon; | −0.548976 |
| | | | | CD40; | | | | | | 1stExon; | |
| | | | | CD40; | | | | | | 5′UTR; | |
| | | | | CD40 | | | | | | 5′UTR | |
| 151 | CD40 | Stimulatory | 12 | CD40; | No | cg21601405 | 20 | 44747006 | Island | 1stExon; | −0.63227 |
| | | | | CD40 | | | | | | 1stExon | |
| 152 | CD40 | Stimulatory | 13 | CD40; | No | cg16686951 | 20 | 44747351 | S_Shore | Body; | −0.616846 |
| | | | | CD40 | | | | | | Body | |
| 153 | CD40 | Stimulatory | 14 | CD40; | No | cg06218285 | 20 | 44751033 | S_Shelf | Body; | 0.505468 |
| | | | | CD40 | | | | | | Body | |
| 154 | CD40 | Stimulatory | 15 | CD40; | No | cg07222575 | 20 | 44757985 | | 3′UTR; | 0.481161 |
| | | | | CD40 | | | | | | 3′UTR | |
| 155 | CD70 | Stimulatory | 1 | CD70 | No | cg26737640 | 19 | 6587584 | N_Shelf | Body | 0.356726 |
| 156 | CD70 | Stimulatory | 2 | CD70 | No | cg15679532 | 19 | 6588838 | N_Shore | Body | 0.174149 |
| 157 | CD70 | Stimulatory | 3 | CD70 | No | cg27335924 | 19 | 6588898 | N_Shore | Body | 0.0327536 |
| 158 | CD70 | Stimulatory | 4 | CD70 | No | cg18475039 | 19 | 6590106 | N_Shore | Body | 0.0265233 |
| 159 | CD70 | Stimulatory | 5 | CD70 | No | cg25949886 | 19 | 6590516 | Island | Body | 0.0366861 |
| 160 | CD70 | Stimulatory | 6 | CD70 | No | cg14870229 | 19 | 6590801 | Island | Body | −0.275658 |
| 161 | CD70 | Stimulatory | 7 | CD70 | No | cg24778383 | 19 | 6590895 | Island | 1stExon | −0.141889 |
| 162 | CD70 | Stimulatory | 8 | CD70 | No | cg23263923 | 19 | 6591204 | S_Shore | TSS200 | −0.168459 |
| 163 | CD70 | Stimulatory | 9 | CD70 | No | cg22633597 | 19 | 6591674 | S_Shore | TSS1500 | 0.0722559 |
| 164 | CD70 | Stimulatory | 10 | CD70 | No | cg11904429 | 19 | 6592554 | S_Shore | TSS1500 | 0.0906074 |
| 165 | LIGHT | Stimulatory | 1 | TNFSF14; | No | cg01432753 | 19 | 6664926 | | 3′UTR; | 0.199538 |
| | | | | TNFSF14 | | | | | | 3′UTR | |
| 166 | LIGHT | Stimulatory | 2 | TNFSF14; | No | cg23071186 | 19 | 6669849 | | Body; | −0.246688 |
| | | | | TNFSF14 | | | | | | Body | |
| 167 | LIGHT | Stimulatory | 3 | TNFSF14; | Yes | cg10362335 | 19 | 6670109 | | 5′UTR; | −0.338743 |
| | | | | TNFSF14 | | | | | | 5′UTR | |
| 168 | LIGHT | Stimulatory | 4 | TNFSF14; | Yes | cg04891836 | 19 | 6670390 | | 1stExon; | −0.216417 |
| | | | | TNFSF14; | | | | | | 5′UTR; | |
| | | | | TNFSF14; | | | | | | 5′UTR; | |
| | | | | TNFSF14 | | | | | | 1stExon | |
| 169 | LIGHT | Stimulatory | 5 | TNFSF14; | No | cg16043888 | 19 | 6670865 | | TSS1500; | 0.0925605 |
| | | | | TNFSF14 | | | | | | TSS1500 | |
| 170 | LIGHT | Stimulatory | 6 | TNFSF14; | No | cg05348870 | 19 | 6671045 | | TSS1500; | 0.15579 |
| | | | | TNFSF14 | | | | | | TSS1500 | |
| 171 | OX40L | Stimulatory | 1 | TNFSF4 | No | cg15112923 | 1 | 173155033 | | 3′UTR | 0.0275687 |
| 172 | OX40L | Stimulatory | 2 | TNFSF4 | No | cg24633390 | 1 | 173159746 | | Body | 0.316404 |
| 173 | OX40L | Stimulatory | 3 | TNFSF4 | No | cg21876925 | 1 | 173175328 | | Body | −0.196511 |
| 174 | OX40L | Stimulatory | 4 | TNFSF4; | Yes | cg16517394 | 1 | 173176362 | | 5′UTR; | −0.323261 |
| | | | | TNFSF4 | | | | | | 1stExon | |
| 175 | OX40L | Stimulatory | 5 | TNFSF4; | Yes | cg21439763 | 1 | 173176366 | | 5′UTR; | −0.365141 |
| | | | | TNFSF4 | | | | | | 1stExon | |
| 176 | OX40L | Stimulatory | 6 | TNFSF4 | Yes | cg26315984 | 1 | 173176501 | | TSS200 | −0.259014 |
| 177 | OX40L | Stimulatory | 7 | TNFSF4 | Yes | cg10861599 | 1 | 173176523 | | TSS200 | −0.307549 |
| 178 | CD137L | Stimulatory | 1 | TNFSF9 | No | cg15451045 | 19 | 6529614 | N_Shore | TSS1500 | 0.0524961 |
| 179 | CD137L | Stimulatory | 2 | TNFSF9 | No | cg26444348 | 19 | 6530187 | N_Shore | TSS1500 | 0.0171351 |
| 180 | CD137L | Stimulatory | 3 | TNFSF9; | Yes | cg01186777 | 19 | 6531016 | Island | 5′UTR; | −0.351878 |
| | | | | TNFSF9 | | | | | | 1stExon | |
| 181 | CD137L | Stimulatory | 4 | TNFSF9 | No | cg05670459 | 19 | 6531335 | Island | Body | −0.15091 |
| 182 | CD137L | Stimulatory | 5 | TNFSF9 | No | cg00169167 | 19 | 6531539 | Island | Body | −0.189517 |
| 183 | CD137L | Stimulatory | 6 | TNFSF9 | No | cg24342464 | 19 | 6532277 | S_Shore | Body | 0.0195071 |
| 184 | CD137L | Stimulatory | 7 | TNFSF9 | No | cg20617093 | 19 | 6532849 | N_Shore | Body | 0.158907 |
| 185 | CD137L | Stimulatory | 8 | TNFSF9 | No | cg03907016 | 19 | 6534760 | Island | Body | 0.442548 |
| 186 | CD137L | Stimulatory | 9 | TNFSF9 | No | cg14995475 | 19 | 6534774 | Island | Body | 0.46594 |
| 187 | CD137L | Stimulatory | 10 | TNFSF9 | No | cg10632765 | 19 | 6534916 | Island | Body | 0.493373 |
| 188 | CD137L | Stimulatory | 11 | TNFSF9 | No | cg21759280 | 19 | 6534936 | Island | Body | 0.466407 |
| 189 | CD137L | Stimulatory | 12 | TNFSF9 | No | cg10818587 | 19 | 6535078 | Island | 3′UTR | 0.391184 |
| 190 | GITRL | Stimulatory | 1 | TNFSF18 | No | cg18879481 | 1 | 1 | | Body | 0.0672151 |
| 191 | GITRL | Stimulatory | 2 | TNFSF18 | No | cg05335876 | 1 | 173018989 | | Body | 0.0667748 |
| 192 | GITRL | Stimulatory | 3 | TNFSF18 | No | cg19589427 | 1 | 173019720 | | Body | 0.0586345 |
| 193 | GITRL | Stimulatory | 4 | TNFSF18 | No | cg05936800 | 1 | 173020492 | | TSS1500 | 0.0327653 |
| 194 | GITRL | Stimulatory | 5 | TNFSF18 | No | cg11532054 | 1 | 173021195 | | TSS1500 | 0.0303499 |
| 195 | B7RP1 | Stimulatory | 1 | ICOSLG | No | cg23813082 | 21 | 45648328 | | 3′UTR | 0.22599 |
| 196 | B7RP1 | Stimulatory | 2 | ICOSLG | No | cg25726128 | 21 | 45656979 | N_Shelf | Body | 0.19852 |
| 197 | B7RP1 | Stimulatory | 3 | ICOSLG | No | cg03048921 | 21 | 45658538 | N_Shore | Body | 0.160248 |
| 198 | B7RP1 | Stimulatory | 4 | ICOSLG | No | cg06033443 | 21 | 45658683 | N_Shore | Body | 0.193176 |
| 199 | B7RP1 | Stimulatory | 5 | ICOSLG | No | cg13520931 | 21 | 45660251 | Island | Body | 0.0226509 |
| 200 | B7RP1 | Stimulatory | 6 | ICOSLG | No | cg17249942 | 21 | 45660288 | Island | Body | 0.0275158 |
| 201 | B7RP1 | Stimulatory | 7 | ICOSLG; | No | cg06173626 | 21 | 45660806 | Island | 1stExon; | 0.0348772 |
| | | | | ICOSLG | | | | | | 5′UTR | |
| 202 | B7RP1 | Stimulatory | 8 | ICOSLG; | No | cg04256691 | 21 | 45660826 | Island | 1stExon; | 0.0359561 |
| | | | | ICOSLG | | | | | | 5′UTR | |
| 203 | B7RP1 | Stimulatory | 9 | ICOSLG | No | cg24327461 | 21 | 45661066 | Island | TSS1500 | 0.0161379 |
| 204 | B7RP1 | Stimulatory | 10 | ICOSLG | No | cg04112169 | 21 | 45661261 | Island | TSS1500 | −0.122396 |
| 205 | B7RP1 | Stimulatory | 11 | ICOSLG | No | cg00993674 | 21 | 45661322 | Island | TSS1500 | −0.13978 |
| 206 | B7RP1 | Stimulatory | 12 | ICOSLG | No | cg09800026 | 21 | 45662262 | Island | TSS1500 | 0.0685446 |
| 207 | HLA-A | Stimulatory | 1 | HLA-A | No | cg11086883 | 6 | 29908891 | N_Shore | TSS1500 | −0.135477 |
| 208 | HLA-A | Stimulatory | 2 | HLA-A | Yes | cg00390191 | 6 | 29908976 | N_Shore | TSS1500 | −0.251201 |
| 209 | HLA-A | Stimulatory | 3 | HLA-A | No | cg07163603 | 6 | 29910051 | N_Shore | TSS1500 | −0.137586 |
| 210 | HLA-A | Stimulatory | 4 | HLA-A | Yes | cg05523662 | 6 | 29910101 | N_Shore | TSS1500 | −0.334206 |
| 211 | HLA-A | Stimulatory | 5 | HLA-A | Yes | cg19151378 | 6 | 29910146 | N_Shore | TSS200 | −0.308148 |
| 212 | HLA-A | Stimulatory | 6 | HLA-A | Yes | cg20142377 | 6 | 29910206 | Island | TSS200 | −0.238395 |
| 213 | HLA-A | Stimulatory | 7 | HLA-A | Yes | cg20879959 | 6 | 29910208 | Island | TSS200 | −0.254067 |
| 214 | HLA-A | Stimulatory | 8 | HLA-A | Yes | cg25291387 | 6 | 29910237 | Island | TSS200 | −0.290732 |
| 215 | HLA-A | Stimulatory | 9 | HLA-A | Yes | cg15319255 | 6 | 29910269 | Island | TSS200 | −0.259475 |
| 216 | HLA-A | Stimulatory | 10 | HLA-A | Yes | cg17678719 | 6 | 29910273 | Island | TSS200 | −0.261888 |
| 217 | HLA-A | Stimulatory | 11 | HLA-A | Yes | cg23489273 | 6 | 29910292 | Island | TSS200 | −0.206572 |
| 218 | HLA-A | Stimulatory | 12 | HLA-A | No | cg05157171 | 6 | 29910411 | Island | Body | −0.125061 |
| 219 | HLA-A | Stimulatory | 13 | HLA-A | No | cg24580035 | 6 | 29910525 | Island | Body | −0.219747 |
| 220 | HLA-A | Stimulatory | 14 | HLA-A | No | cg11808100 | 6 | 29910755 | Island | Body | −0.186328 |
| 221 | HLA-A | Stimulatory | 15 | HLA-A | No | cg25548869 | 6 | 29910776 | Island | Body | −0.392067 |
| 222 | HLA-A | Stimulatory | 16 | HLA-A | No | cg19748509 | 6 | 29910778 | Island | Body | −0.28115 |
| 223 | HLA-A | Stimulatory | 17 | HLA-A | No | cg09803951 | 6 | 29910796 | Island | Body | −0.104342 |
| 224 | HLA-A | Stimulatory | 18 | HLA-A | No | cg22951229 | 6 | 29910912 | Island | Body | −0.31346 |
| 225 | HLA-A | Stimulatory | 19 | HLA-A | No | cg21591486 | 6 | 29910994 | Island | Body | −0.299429 |
| 226 | HLA-A | Stimulatory | 20 | HLA-A | No | cg05738749 | 6 | 29911004 | Island | Body | −0.352269 |
| 227 | HLA-A | Stimulatory | 21 | HLA-A | No | cg11722179 | 6 | 29911011 | Island | Body | −0.234728 |
| 228 | HLA-A | Stimulatory | 22 | HLA-A | No | cg18106971 | 6 | 29911028 | Island | Body | −0.254845 |
| 229 | HLA-A | Stimulatory | 23 | HLA-A | No | cg10886493 | 6 | 29911036 | Island | Body | −0.234664 |
| 230 | HLA-A | Stimulatory | 24 | HLA-A | No | cg18599206 | 6 | 29911087 | Island | Body | −0.471095 |
| 231 | HLA-A | Stimulatory | 25 | HLA-A | No | cg19045970 | 6 | 29911091 | Island | Body | −0.415368 |
| 232 | HLA-A | Stimulatory | 26 | HLA-A | No | cg05839762 | 6 | 29911095 | Island | Body | −0.436049 |
| 233 | HLA-A | Stimulatory | 27 | HLA-A | No | cg14772439 | 6 | 29911104 | Island | Body | −0.419053 |
| 234 | HLA-A | Stimulatory | 28 | HLA-A | No | cg10018004 | 6 | 29911261 | Island | Body | −0.113211 |
| 235 | HLA-A | Stimulatory | 29 | HLA-A | No | cg14018363 | 6 | 29911265 | Island | Body | −0.144475 |
| 236 | HLA-A | Stimulatory | 30 | HLA-A | No | cg09535358 | 6 | 29911295 | Island | Body | −0.231793 |
| 237 | HLA-A | Stimulatory | 31 | HLA-A | No | cg08039587 | 6 | 29911334 | Island | Body | −0.414116 |
| 238 | HLA-A | Stimulatory | 32 | HLA-A | No | cg00082981 | 6 | 29911339 | Island | Body | −0.309379 |
| 239 | HLA-A | Stimulatory | 33 | HLA-A | No | cg16742075 | 6 | 29911366 | Island | Body | −0.499466 |
| 240 | HLA-A | Stimulatory | 34 | HLA-A | No | cg20408505 | 6 | 29911494 | S_Shore | Body | −0.518677 |
| 241 | HLA-A | Stimulatory | 35 | HLA-A | No | cg25637655 | 6 | 29911542 | S_Shore | Body | −0.520002 |
| 242 | HLA-A | Stimulatory | 36 | HLA-A | No | cg17608381 | 6 | 29911550 | S_Shore | Body | −0.494328 |
| 243 | HLA-A | Stimulatory | 37 | HLA-A | No | cg11946459 | 6 | 29911558 | S_Shore | Body | −0.503476 |
| 244 | HLA-A | Stimulatory | 38 | HLA-A | No | cg23303505 | 6 | 29911836 | S_Shore | Body | −0.238288 |
| 245 | HLA-A | Stimulatory | 39 | HLA-A | No | cg18349863 | 6 | 29912713 | S_Shore | Body | 0.0608357 |
| 246 | HLA-A | Stimulatory | 40 | HLA-A | No | cg20221094 | 6 | 29913098 | S_Shore | Body | 0.0494603 |
| 247 | HLA-A | Stimulatory | 41 | HLA-A | No | cg19585676 | 6 | 29913343 | S_Shore | 3′UTR | 0.0965819 |

The probeID, the location within the gene locus, the R correlation coefficient between probe β-value and gene expression for all probes used in the study are summarized.

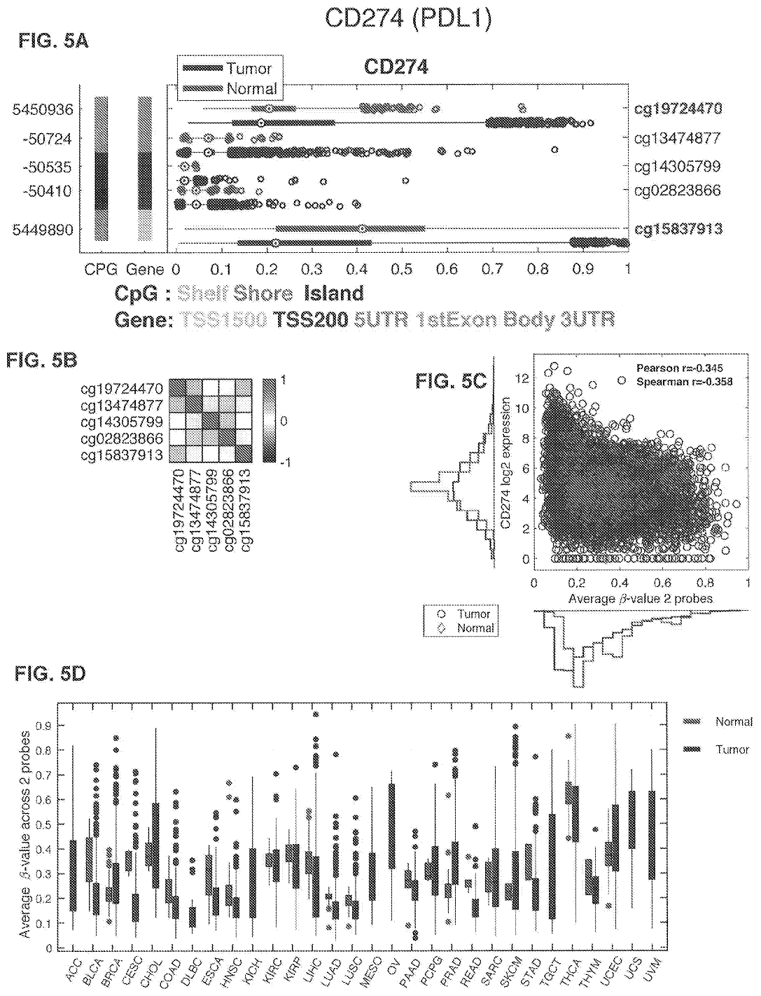

These results indicate that the tumor-immune synapse is regulated by methylation in cancer. Sporadic evidence for regulation of HLA, CD40, or CD80 by methylation in select tumor types now appears a more generalized phenomenon in the majority of co-stimulatory and immune checkpoint genes across tumor types. Interestingly, two probes within the promoter region were negatively correlated with the gene expression. However, a clear trend for hypomethylation of PD-L1 locus in comparison to normal adjacent tissue was not observed, indicating competing mechanisms governing PD-L1 expression (FIG. 5).

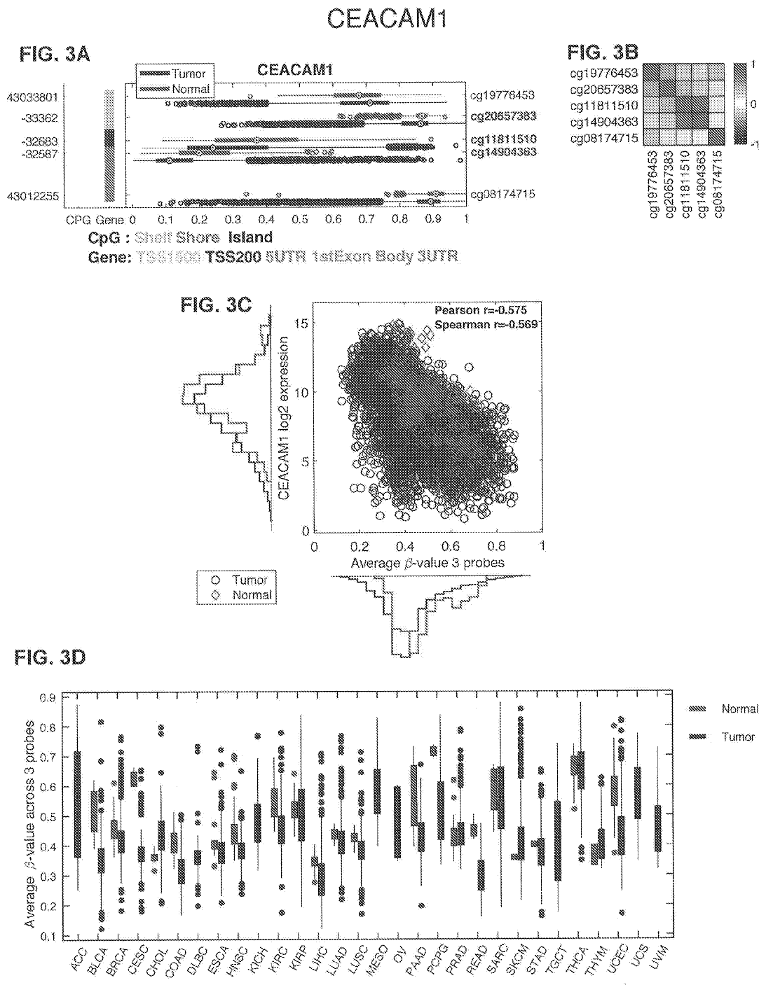

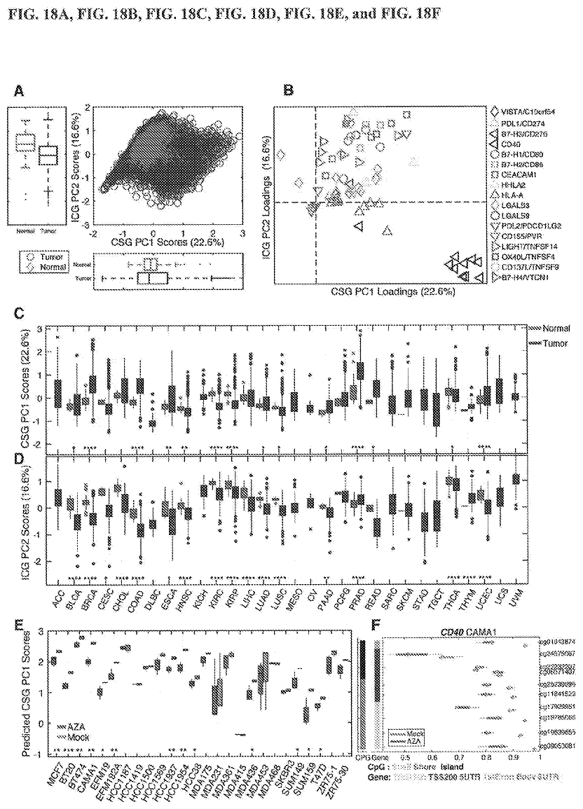

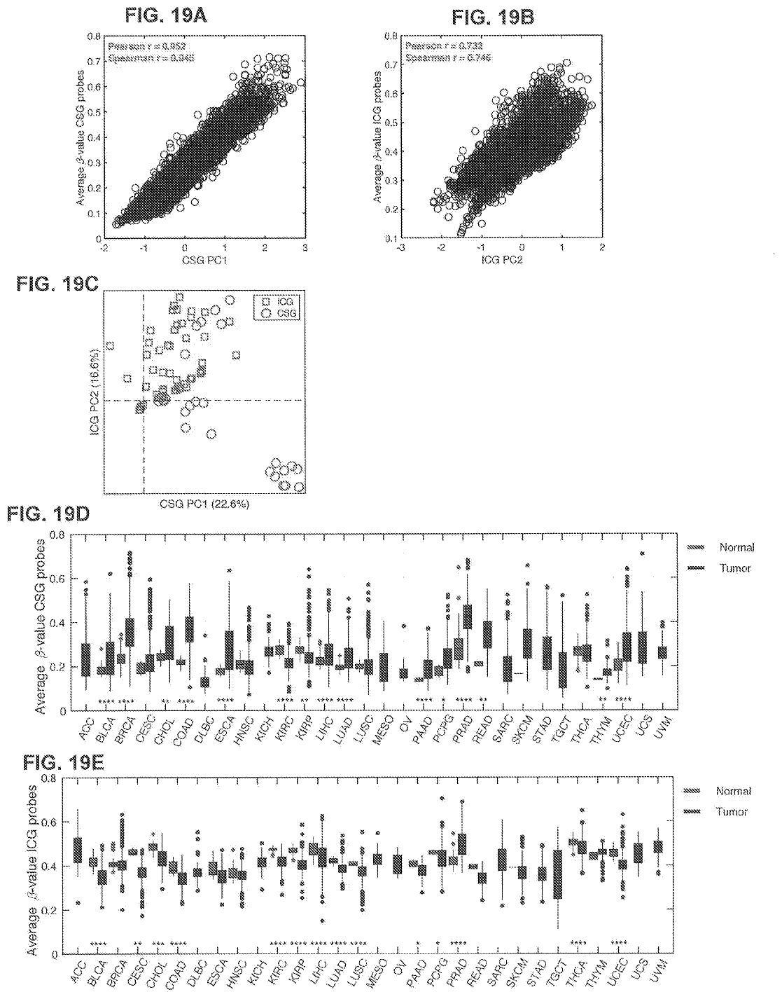

Next, we conducted a principal component analysis (PCA) to summarize the methylation pattern across all genes and their CpG-probes. To minimize noise and enrich for biologically relevant signal, only the CSGs and ICGs CpG-probes that demonstrated negative correlation (r<−0.2) between the methylation status and their corresponding gene expression and located in the TSS1500, TSS200, 5′UTR regions were selected for further analysis; in total 75 probes. (FIG. 3-17, Table 3-4). PCA revealed two major principal components (PCs), explaining 22.6% and 16.6% of the variation, respectively. A two-dimensional representation of PC1 and PC2 for 8,186 solid tumors and 745 normal adjacent tissues clearly showed that many tumors have an abnormal methylation pattern (FIG. 18A). Strikingly, the dominant components of PC1 were CSGs, in particular CD40 and HLA-A. By contrast, PC2 was mainly driven by ICGs including VTCN1, HHLA2, PDL1, CEACAM1, CD80, and CD86 (FIG. 18B, 19). Consequently, PC1 and PC2 were highly correlated with average β-values of CSG probes and ICG probes respectively (FIG. 18B, 19A-C). Probes from the same gene generally clustered together further confirming robustness of this analysis (FIG. 18B). It should be noted that all CpG-probes contribute to both PCA components with variable contributions, some with a negative weight for a specific PCA component. The total score for a sample is thus a weighted average of all variables. Consistent with the methylation patterns observed with individual CSG and ICG, primary tumor exhibited higher PC1 and lower PC2 scores in comparison to the normal adjacent tissue score across disease sites (FIG. 18C-D), which was also replicated in the average β-values of CSG and ICG probes (FIG. 19D-E). Importantly, we observed reversal of hypermethylation of CSGs by 5-azacytidine in the dataset of 26 epithelial cancer cell lines with a significant decrease in PC1 scores (FIG. 18E). At an individual gene level, demethylation of CD40 by azacytidine was also evident (FIG. 18F) underscoring that the methylation status of CSGs is therapeutically actionable.

TABLE 4
List of 75 selected probes for PCA.
ProbeId
cg14904363
cg11811510
cg20657383
cg27625456
cg21157094
cg15837913
cg19724470
cg07211259
cg06655361
cg10586317
cg14910296
cg09706277
cg16408593
cg15597855
cg24006253
cg04718492
cg20821424
cg01436254
cg16331599
cg13617155
cg13458803
cg21572897
cg02470871
cg12978275
cg02124498
cg08817540
cg02059214
cg10431989
cg01396723
cg04566018
cg14538146
cg10777702
cg07917289
cg05012825
cg04306507
cg20008101
cg26335127
cg02183170
cg18996663
cg13185030
cg19099850
cg13570982
cg17403875
cg09939831
cg19899505
cg18273401
cg04260307
cg14871010
cg23575099
cg09053081
cg19839655
cg19785066
cg17929951
cg11841529
cg25239996
cg06571407
cg22232207
cg24575067
cg01943874
cg10362335
cg04891836
cg16517394
cg21439763
cg26315984
cg10861599
cg01186777
cg00390191
cg05523662
cg19151378
cg20142377
cg20879959
cg25291387
cg15319255
cg17678719
cg23489273
The probes located within TSS1500, TSS200, 5’UTR within a gene with negative correlation to its expression are selected for PCA.

Two-dimensional evaluation of CSG and ICG methylation status revealed that normal tissues generally exhibit relative hypermethylation of ICGs and hypomethylation of CSGs, demonstrating absence of epigenetic brake to suppress immune response. Indeed, highly efficient central tolerance mechanisms governing clonal deletion of self-reactive T-cells allows normal tissues to remain highly immunogenic to any abnormal presence of foreign antigens, which usually represent infection. By contrast, tumor tissues manifest either hypermethylation of CSGs and/or hypomethylation of ICGs, effectively employing epigenetic mechanisms to deliberately suppress the immune system. Because of neo-antigens, oncogenic viral antigens, or cancer testis antigens, tumor specific immune responses ensue. Therefore, altered methylation status reflects tumor adaptation to evolutionary pressure exerted by immune-surveillance. Relatively consistent methylation phenotype between early stage and late stage melanoma indicates such epigenetic adaptation occurs early during carcinogenesis, which explains in part the markedly consistent methylation phenotype of immune synapse genes across tumor types. While expression of HLA and co-stimulatory/immune checkpoint molecules is frequently dysregulated in cancer via multiple mechanisms, heritable changes to impact the entire tumor tissue as a whole require the initial cascade of tolerogenic signal to involve genetic or epigenetic changes. Because germline or somatic mutations of these immune synapse genes are rare events, the immune status of tumor manifest on the epigenetic footprints of immune synapse genes.

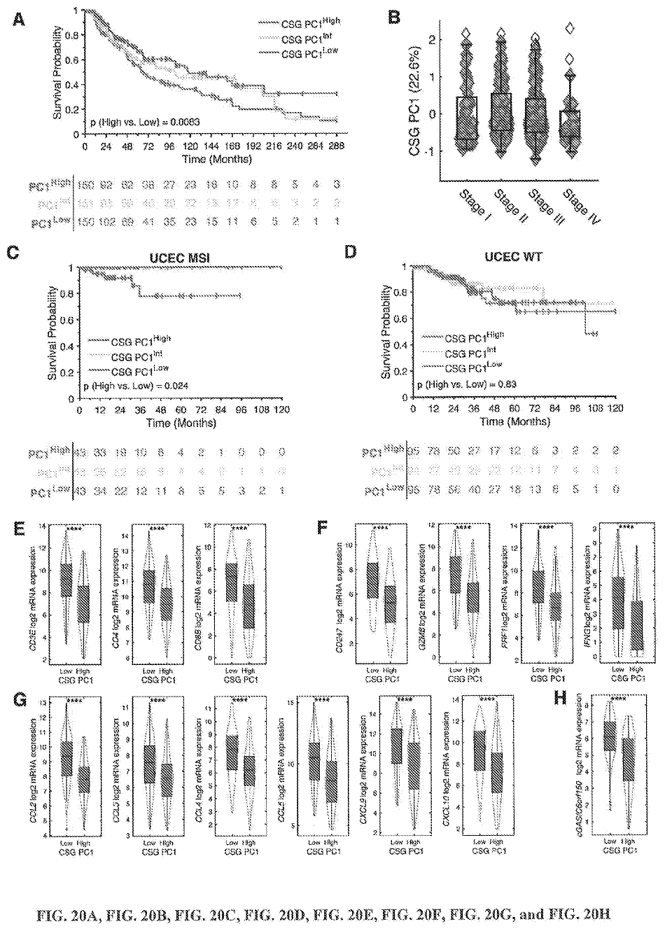

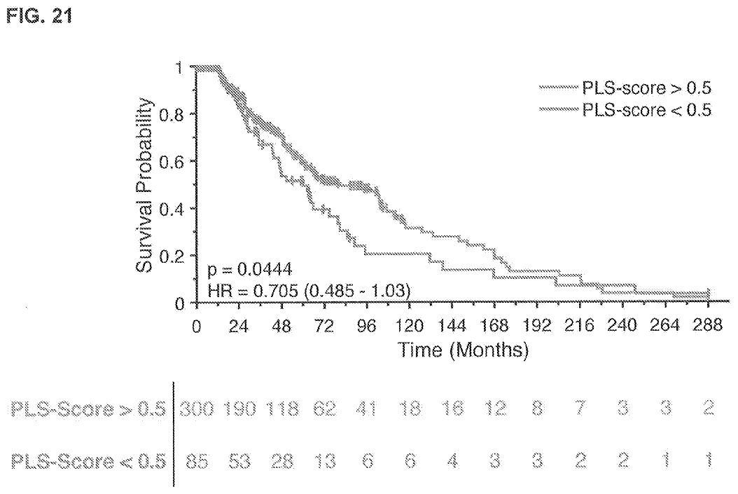

Because immune evasion is critical for cancer progression and survival, we hypothesized that the differential methylation status of the immune synapse genes can determine clinical outcome. Therefore, we investigated the clinical relevance of the PCA model in melanoma, a prototypic immunogenic cancer. PC1 was a determinant of disease specific survival (DSS) in melanoma with significant survival advantage in PC1^{low} patients characterized by hypomethylation of CSGs (FIG. 20A). An alternate approach with partial least squares (PLS) modeling using the outcome as response variable also confirmed differences in survival outcome based on CSGs (FIG. 21). Interestingly, the PC1 score was relatively consistent among early and late stage melanoma patients, and thus, the survival difference was independent of patient staging (FIG. 20B).

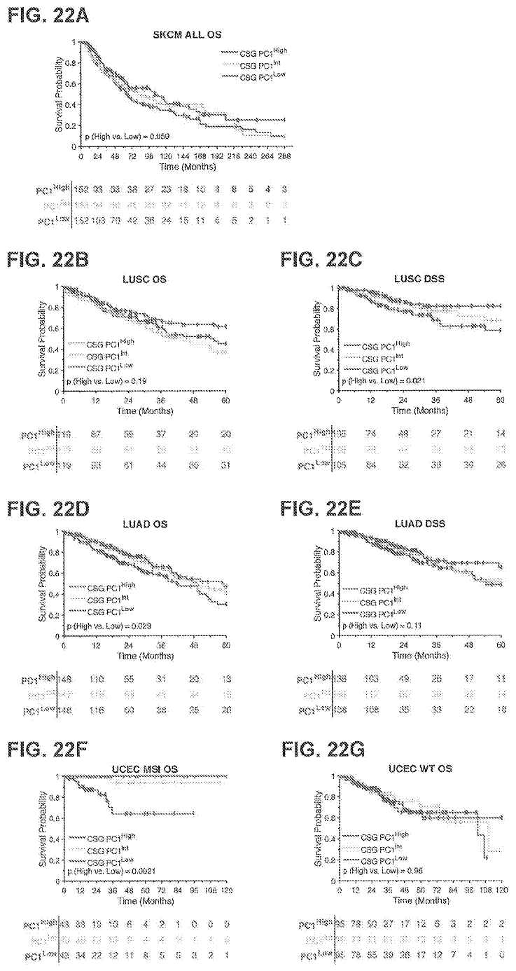

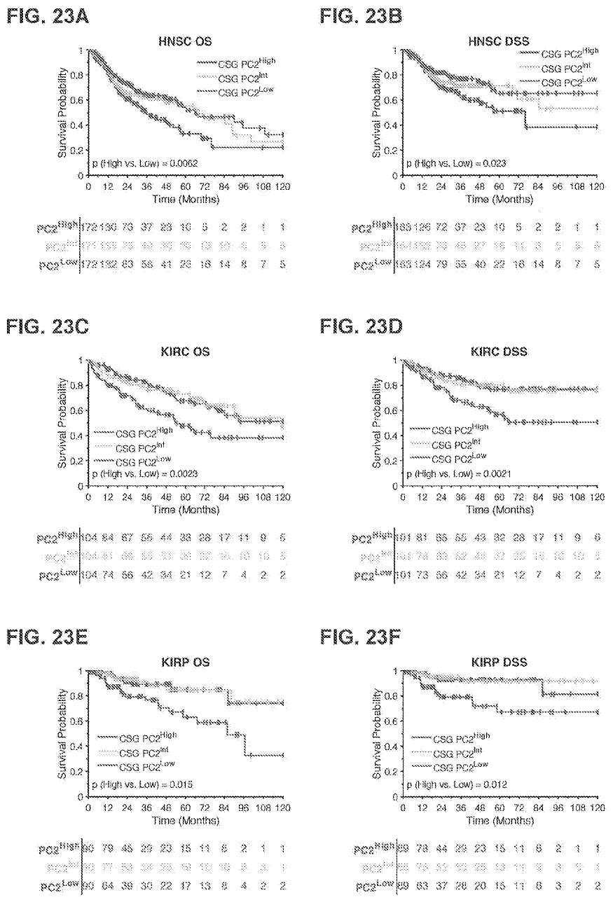

The methylation status of immune synapse genes was prognostic only in immunogenic tumors indicating that modulation of tumor-immune synapse by methylation can become clinically relevant only in the presence of active anti-tumor immune responses. For instance, PC1 was prognostic for DSS in uterine corpus endometrial carcinoma (UCEC) with microsatellite instability (MSI-H) (FIG. 20C). By contrast, no differences in survival was noted based on PC1 in UCEC without MSI (WT) (FIG. 20D). Consistently, the methylation status correlated with overall survival (OS) and DSS also in other relatively immunogenic cancers, including non-small cell lung cancer (NSCLC), renal cell carcinoma (RCC) and head and neck cancer (HNSC). Similar to the findings with melanoma, NSCLC patients with lower PC1 score demonstrated improved survival (FIG. 22). By contrast, prognosis for head and neck squamous cell carcinoma and renal cell carcinoma correlated with PC2 (FIG. 23).

Increased tumor infiltration by CD4⁺ and CD8⁺ T-cells was evident in PC1^{low} patients (FIG. 20E). Further, increased levels of CD3ξ (CD247), Granzyme B (GZMB), Perforin (PRF1), and IFNγin PC1^{low} patients indicate superior effector functions by these T-cells (FIG. 20F). Interestingly, key chemokines that drive T-cell recruitment and trafficking in melanoma, CCL2, CCL3, CCL4, CCL5, CXCL9, and CXCL10, were elevated in PC1^{low} patients (FIG. 20G). More recently, STING/cGAS pathway has been critically implicated in tumor immunogenicity. A significant increase in cGAS expression was also manifest in PC1^{low} patients (FIG. 20H). Therefore, hypomethylation of CSGs in melanoma was associated with improved survival as well as enhanced tumor immunogenicity and recruitment of effector T-cells.

In summary, we report methylation of immune synapse genes as a crucial driver of tolerogenic immune landscapes in cancer. Notably, preclinical studies have demonstrated the efficacy of demethylating agents to augment immunotherapy. Based on this study, we show that the subset of patients with hypermethylated CSGs (PC1^{high}) benefit from combination therapy of PD1 blockade with 5-azacitidine, while conversely, patients with hypermethylated ICGs (PC2^{high}) can be adversely impacted. Given negative findings from the phase II randomized clinical trial of oral 5-azacitidine plus pembrolizumab vs pembrolizumab plus placebo, patient selection can be crucial to overcome resistance to PD1 blockade. Alternatively, targeted editing of tumor methylation of immune synapse genes by TET1 or DNMT3a via CRISPR-cas allows for a personalized approach to augment immunotherapy. Notably, the methylation status of immune synapse genes can be utilized to predict response to immunotherapy. The major advantage to the use of the methylation status is that DNA is stable and degradation is less likely in Formalin-Fixed Paraffin-Embedded tissues, and thus anticipated to be more robust than RNA based or histology based approaches.

b) Methods

(1) Analysis of TCGA Methylation Database

TCGA Level 1 IDAT files for the selected tumor types was downloaded between April and May of 2016 using the Data Matrix. Preprocessing the data included normalization via internal controls probe followed by background subtraction using the methylumi R package from Bioconductor. The calculated β-values were then extracted from the MethyLumiSet object following preprocessing.

(2) Analysis of TCGA RNAseq Database

The TCGA RNAseq samples was extracted from the “EBPlusPlusAdjustPANCAN_IlluminaHiSeq_RNASeqV2.geneExp.tsv” file and log2 transformed, log2(x+1).

(3) GSE57342 5-azacitidine Treated Cancer Cell Lines

The GSE57342 processed dataset was downloaded and cell lines with more than three Mock- and three 5-azacitidine-treated samples was selected for analysis.

(4) T-SNE Analysis

T-SNE was calculated using all 247 probes for the selected 20 genes across all TCGA samples. The 50 first PCA-components was used as input with perplexity=50 and Euclidian distance as implemented in MATLAB.

(5) Correlation Coefficient Heatmap

The Pearson's correlation coefficients between all the probes within a gene were calculated and displayed as a heatmap.

(6) Principal Component Analysis

We used the first and second principal component (a weighted average β-values among the CSG and ICG probes), as they account for the largest variability in the data, to represent the overall methylation status for 8,931 tumor and normal samples in the TCGA database. That is, PC=Σwᵢxᵢ, a weighted average β-values among the selected CSG and ICG probes, where xi represents gene i β-value, wᵢ is the corresponding weight (loading coefficient) with Σwᵢ²=1, and the wᵢ values maximize the variance of Σwᵢxᵢ. For each gene, a set of probes were selected using the following criteria to minimize noise, r<−0.2 (methylation vs gene expression) located in the TSS1500, TSS200 or the 5′UTR (Table 4). Each probe was centered but not scaled before PCA calculations.

(7) Survival Analysis

Tertiles was used to define high, intermediate (Int) and low PC1 or PC2 for melanoma, NSCLC, HNSC, RCC, UCEC MSI^{hi} and wild type patients. Kaplan-Meier curves were then plotted based on tertile scores.

(8) Partial Least Squares (PLS) Modelling

A PLS model was derived using melanoma poor survivors (DSS Dead<12 months, 0) and long survivors (DSS Alive>120 months, 1) as a binary response using the CSG-probes. Cross validation indicated two significant PLS components. The PLS model was then applied to the melanoma samples not used in training. Samples with a predicted response>0.5 was compared to samples with a predicted response<0.5 using a log rank test.

(9) MSI Status

Samples with a MANTIS score larger than 0.4 was considered MSI positive.

(10) Statistics

T-SNE, PCA, PLS, Pearson's correlation statistics, and two-sided Student's t-tests were done in MATLAB R2018B. Survival analysis was done using MatSurv.

### D. References

Bonneville R, Krook M A, Kautto E A, Miya J, Wing M R, Chen H Z, et al. Landscape of Microsatellite Instability Across 39 Cancer Types. JCO precision oncology. 2017; 2017.
Chen L, and Flies D B. Molecular mechanisms of T-cell co-stimulation and co-inhibition. Nature reviews Immunology. 2013; 13(4):227-42.
Chiappinelli K B, Strissel P L, Desrichard A, Li H, Henke C, Akman B, et al. Inhibiting DNA Methylation Causes an Interferon Response in Cancer via dsRNA Including Endogenous Retroviruses. Cell. 2017; 169(2):361.
Davis S D P, Bilke S, Triche T, Bootwalla M. Handle Illumina methylation data. R package version 2.12.0. 2014.
Ehrlich M. DNA methylation and cancer-associated genetic instability. Adv Exp Med Biol. 2005;570:363-92.
Fitzpatrick D R, and Wilson C B. Methylation and demethylation in the regulation of genes, cells, and responses in the immune system. Clin Immunol. 2003; 109(1):37-45.
Harlin H, Meng Y, Peterson A C, Zha Y, Tretiakova M, Slingluff C, et al. Chemokine expression in melanoma metastases associated with CD8+ T-cell recruitment. Cancer Res. 2009; 69(7):3077-85.
Kluger H M, Zito C R, Turcu G, Baine M K, Zhang H, Adeniran A, et al. PD-L1 Studies Across Tumor Types, Its Differential Expression and Predictive Value in Patients Treated with Immune Checkpoint Inhibitors. Clinical cancer research: an official journal of the American Association for Cancer Research. 2017; 23(15):4270-9.
Kowanetz M, Zou W, Gettinger S N, Koeppen H, Kockx M, Schmid P, et al. Differential regulation of PD-L1 expression by immune and tumor cells in NSCLC and the response to treatment with atezolizumab (anti-PD-L1). Proc Natl Acad Sci USA. 2018; 115(43):E10119-E26.
Li H, Chiappinelli K B, Guzzetta A A, Easwaran H, Yen R W, Vatapalli R, et al Immune regulation by low doses of the DNA methyltransferase inhibitor 5-azacitidine in common human epithelial cancers. Oncotarget. 2014; 5(3):587-98.
Micevic G, Thakral D, McGeary M, and Bosenberg M. PD-L1 methylation regulates PD-L1 expression and is associated with melanoma survival. PigmenT-cell & melanoma research. 2018.
Pardon D M. The blockade of immune checkpoints in cancer immunotherapy. Nature reviews. 2012; 12(4):252-64.
Sade-Feldman M, Jiao Y J, Chen J H, Rooney M S, Barzily-Rokni M, Eliane J P, et al. Resistance to checkpoint blockade therapy through inactivation of antigen presentation. Nature communications. 2017; 8(1):1136.
Scarpa M, Scarpa M, Castagliuolo I, Erroi F, Basato S, Brun P, et al. CD80 down-regulation is associated to aberrant DNA methylation in non-inflammatory colon carcinogenesis. BMC cancer. 2016; 16:388.
Serrano A, Tanzarella S, Lionello I, Mendez R, Traversari C, Ruiz-Cabello F, et al. Rexpression of HLA class I antigens and restoration of antigen-specific CTL response in melanoma cells following 5-aza-2′-deoxycytidine treatment. International journal of cancer Journal international du cancer. 2001; 94(2):243-51.
Suzuki M M, and Bird A. DNA methylation landscapes: provocative insights from epigenomics. Nature reviews Genetics. 2008; 9(6):465-76.
Thorsson V, Gibbs D L, Brown S D, Wolf D, Bortone D S, Ou Yang T H, et al. The Immune Landscape of Cancer. Immunity. 2018; 48(4):812-30 e14.
Tirapu I, Huarte E, Guiducci C, Arina A, Zaratiegui M, Murillo O, et al. Low surface expression of B7-1 (CD80) is an immunoescape mechanism of colon carcinoma. Cancer Res. 2006; 66(4):2442-50.
Van der Maaten L. Visualizing Data using t-SNE. Journal of Machine Learning Research. 2008; 9:2579-605.
Wrangle J, Wang W, Koch A, Easwaran H, Mohammad H P, Vendetti F, et al. Alterations of immune response of Non-Small Cell Lung Cancer with Azacytidine. Oncotarget. 2013; 4(11):2067-79.

1. A method of treating, inhibiting, reducing, ameliorating, and/or preventing an immunogenic cancer or metastasis in a subject comprising:
a. obtaining a tissue sample from the subject;
b. assaying the amount of methylation of one or more co-stimulatory genes and/or one or more immune checkpoint genes in the tissue sample; and
c. administering to the subject an immunotherapy wherein an increase in the methylation of one or more co-stimulatory genes relative to a normal control tissue is detected and/or a decrease in the methylation of one or more immune checkpoint genes relative to a normal control tissue is detected.

2. The method of treating, inhibiting, reducing, ameliorating, and/or preventing an immunogenic cancer or metastasis claim 1, wherein the one or more co-stimulatory gene comprises cluster of differentiation (CD) 40 (CD40), CD70, homologous to lymphotoxin, exhibits inducible expression and competes with HSV glycoprotein D for binding to herpesvirus entry mediator, a receptor expressed on T lymphocytes (LIGHT), OX40L, CD137L (4-1BBL), glucocorticoid-induced tumour-necrosis-factor-receptor-related protein (GITR) ligand (GITRL), B7 related protein 1 (B7RP1), and/or human leukocyte antigen (HLA)-A (HLA-A).

3. The method of treating, inhibiting, reducing, ameliorating, and/or preventing an immunogenic cancer or metastasis claim 1, wherein the one or more immune checkpoint gene comprises carcinoembryonic antigen-related adhesion molecule (CEACAM) 1 (CEACAM1), Galectin 9, programmed death ligand (PDL) 1 (PDL1), PDL2, V-domain Ig suppressor of T cell activation (VISTA), B7-H3, B7-H4, B7-2 (CD86), B7-1 (CD80), HHLA2, CD155, and/or Galectin 3.

4. The method treating, inhibiting, reducing, ameliorating, and/or preventing an immunogenic cancer or metastasis of claim 1, wherein the immunotherapy comprises an antibody, cytokine, natural killer (NK) cell, chimeric antigen receptor (CAR) T cell, CAR NK cell, tumor infiltrating lymphocyte (TIL), marrow infiltrating lymphocyte (MIL), and/or tumor infiltrating NK cell (TINK).

5. The method treating, inhibiting, reducing, ameliorating, and/or preventing an immunogenic cancer or metastasis of claim 1, wherein the antibody comprises an immune checkpoint inhibitor blockade.

6. The method treating, inhibiting, reducing, ameliorating, and/or preventing an immunogenic cancer or metastasis of claim 1, further comprising administering to the subject an inhibitor of methylation when the amount of methylation of the one or more co-stimulatory genes is increased relative to a control.

7. The method treating, inhibiting, reducing, ameliorating, and/or preventing an immunogenic cancer or metastasis of claim 6, wherein the inhibitor of methylation comprises azacytidine, decitabine, and/or zebularine.

8. The method of treating, inhibiting, reducing, ameliorating, and/or preventing an immunogenic cancer or metastasis of claim 1, wherein the cancer comprises adenocarcinoma, breast cancer, bladder cancer, cervical cancer, colon cancer, lymphoma, esophageal cancer, renal cancer, lung cancer, mesothelioma, head and neck cancer, cholangiocarcinoma, liver cancer, ovarian cancer, pancreatic cancer, prostate cancer, adrenal gland cancer, nerve cell cancer, rectal cancer, melanoma, sarcoma, testicular cancer, thyroid cancer, uterine cancer, or ocular cancer.

9. The method of treating, inhibiting, reducing, ameliorating, and/or preventing an immunogenic cancer or metastasis of claim 8, wherein the cancer comprises adrenocortical carcinoma, bladder urothelial carcinoma, breast invasive carcinoma, cervical squamous cell carcinoma and endocervical adenocarcinoma, cholangiocarcinoma, colon adenocarcinoma, lymphoid neoplasm diffuse large B-cell lymphoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ sell tumors, thyroid carcinoma, thymoma, uterine corpus endometrial carcinoma, uterine carcinosarcoma, or uveal melanoma.

10. The method of treating, inhibiting, reducing, ameliorating, and/or preventing an immunogenic cancer or metastasis of claim 1, wherein methylation is measured by performing principal component (PC) analysis (PCA) of the one or more co-stimulatory genes and/or one or more immune checkpoint genes; wherein PC^{high} indicates an increase in methylation and PC^{low} indicates a decrease in methylation.

11. A method of assessing the suitability of an immunotherapy treatment regimen for the treatment an immunogenic cancer or metastasis in a subject comprising:
a. obtaining a tissue sample from the subject; and
b. assaying the amount of methylation of one or more co-stimulatory genes and/or one or more immune checkpoint genes in the tissue sample;
wherein an increase in the methylation of one or more co-stimulatory genes relative to a normal control tissue and/or a decrease in the methylation of one or more immune checkpoint genes relative to a normal control tissue indicates that immunotherapy is suitable for treatment of the cancer in the subject.

12. The method of assessing the suitability of an immunotherapy treatment regimen for the treatment of an immunogenic cancer or metastasis in a subject of claim 11, wherein the one or more co-stimulatory gene comprises cluster of differentiation (CD) 40 (CD40), CD70, homologous to lymphotoxin, exhibits inducible expression and competes with HSV glycoprotein D for binding to herpesvirus entry mediator, a receptor expressed on T lymphocytes (LIGHT), OX40L, CD137L (4-1BBL), glucocorticoid-induced tumour-necrosis-factor-receptor-related protein (GITR) ligand (GITRL), B7 related protein 1 (B7RP1), and/or human leukocyte antigen (HLA)-A (HLA-A)

13. The method of assessing the suitability of an immunotherapy treatment regimen for the treatment of an immunogenic cancer or metastasis in a subject of claim 11, wherein the one or more immune checkpoint gene comprises carcinoembryonic antigen-related adhesion molecule (CEACAM) 1 (CEACAM1), Galectin 9, programmed death ligand (PDL) 1 (PDL1), PDL2, V-domain Ig suppressor of T cell activation (VISTA), B7-H3, B7-H4, B7-2 (CD86), B7-1 (CD80), HHLA2, CD155, and/or Galectin 3.

14. The method of assessing the suitability of an immunotherapy treatment regimen for the treatment of an immunogenic cancer or metastasis in a subject of claim 11, wherein the immunotherapy comprises an antibody, cytokine, natural killer (NK) cell, chimeric antigen receptor (CAR) T cell, CAR NK cell, tumor infiltrating lymphocyte (TIL), marrow infiltrating lymphocyte (MIL), and/or tumor infiltrating NK cell (TINK).

15. The method of assessing the suitability of an immunotherapy treatment regimen for the treatment of an immunogenic cancer or metastasis in a subject of claim 11, wherein the antibody comprises an immune checkpoint inhibitor blockade.

16. The method of assessing the suitability of an immunotherapy treatment regimen for the treatment of an immunogenic cancer or metastasis in a subject of claim 11, wherein a decrease in the methylation of one or more co-stimulatory genes relative to a normal control tissue or an increase in the methylation of one or more immune checkpoint genes relative to a normal control indicates that an inhibitor of methylation should not be administered to the subject.

17. The method of assessing the suitability of an immunotherapy treatment regimen for the treatment of an immunogenic cancer or metastasis in a subject of claim 11, wherein an increase in the methylation of one or more co-stimulatory genes relative to a normal control tissue indicates that an inhibitor of methylation can be administered to the subject.

18. The method of assessing the suitability of an immunotherapy treatment regimen for the treatment of an immunogenic cancer or metastasis in a subject of claim 11, wherein the cancer comprises adenocarcinoma, breast cancer, bladder cancer, cervical cancer, colon cancer, lymphoma, esophageal cancer, renal cancer, lung cancer, mesothelioma, head and neck cancer, cholangiocarcinoma, liver cancer, ovarian cancer, pancreatic cancer, prostate cancer, adrenal gland cancer, nerve cell cancer, rectal cancer, melanoma, sarcoma, testicular cancer, thyroid cancer, uterine cancer, or ocular cancer.

19. The method of assessing the suitability of an immunotherapy treatment regimen for the treatment of an immunogenic cancer or metastasis in a subject of claim 18, wherein the cancer comprises adrenocortical carcinoma, bladder urothelial carcinoma, breast invasive carcinoma, cervical squamous cell carcinoma and endocervical adenocarcinoma, cholangiocarcinoma, colon adenocarcinoma, lymphoid neoplasm diffuse large B-cell lymphoma, esophageal carcinoma, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ sell tumors, thyroid carcinoma, thymoma, uterine corpus endometrial carcinoma, uterine carcinosarcoma, or uveal melanoma

20. The method of assessing the suitability of an immunotherapy treatment regimen for the treatment of an immunogenic cancer or metastasis in a subject of claim 11, wherein the assessment is conducted prior to the commencement of any immunotherapy regimen; wherein an increase in the methylation of one or more co-stimulatory genes relative to a normal control tissue and/or a decrease in the methylation of one or more immune checkpoint genes relative to a normal control tissue indicates that the subject can start an immunotherapy regimen; and wherein a decrease in the methylation or same amount of methylation of one or more co-stimulatory genes relative to a normal control tissue and/or an increase in the methylation of one or more immune checkpoint genes relative to a normal control tissue indicates that the subject should start an anti-cancer regimen that is not an immunotherapy.

21. The method of assessing the suitability of an immunotherapy treatment regimen for the treatment of an immunogenic cancer or metastasis in a subject of claim 11, wherein the assessment is conducted after to the commencement of an immunotherapy regimen; wherein an increase in the methylation of one or more co-stimulatory genes relative to a normal control tissue and/or a decrease in the methylation of one or more immune checkpoint genes relative to a normal control tissue indicates that the subject can continue an immunotherapy regimen; and wherein a decrease or same amount of methylation of one or more co-stimulatory genes relative to a normal control tissue and/or an increase or same amount of methylation of one or more immune checkpoint genes relative to a normal control tissue indicates that the subject should discontinue an anti-cancer regimen that is not an immunotherapy.

22. The method of assessing the suitability of an immunotherapy treatment regimen for the treatment of an immunogenic cancer or metastasis in a subject of claim 11, wherein methylation is measured by performing principal component analysis of the one or more co-stimulatory genes and/or one or more immune checkpoint genes; wherein PC^{high} indicates an increase in methylation and PC^{low} indicates a decrease in methylation.
